(19) United States
(12) Patent Application Publication
(10) Pub. No.: US 2015/0291967 A1
(43) Pub. Date: Oct. 15, 2015

(54) COUPLING HERBICIDE RESISTANCE WITH TARGETED INSERTION OF TRANSGENES IN PLANTS

(71) Applicants: Luc MATHIS, (US); Daniel VOYTAS, Falcon Heights, MN (US); Jin LI, Shoreview, MN (US); Feng ZHANG, Plymouth, MN (US); Song LUO, Chicago, IL (US); CELLECTIS, Paris (FR)

(72) Inventors: Luc Mathis, Le Kremlin Bicetre (FR); Daniel F. Voytas, Falcon Heights, MN (US); Jin Li, Shoreview, MN (US); Feng Zhang, Plymouth, MN (US); Song Luo, Chicago, IL (US)

(21) Appl. No.: 14/439,413

(22) PCT Filed: Oct. 31, 2013
(86) PCT No.: PCT/US2013/067744

§ 371 Date: Apr. 29, 2015

Related U.S. Application Data
(60) Provisional application No. 61/720,782, filed on Oct. 31, 2012.

(51) Int. Cl. C12N 15/82

(52) U.S. Cl. CPC C12N 15/8213; C12N 15/8278; C12N 15/8274

(57) ABSTRACT

The present invention relates to methods allowing the targeted insertion of transgenes into a plant genome at desired loci by using homologous recombination combined with rare-cutting endonucleases without the need of inserting an exogenous selectable marker.

### TECHNICAL FIELD

The present invention relates to the field of plant molecular biology. In particular, it relates to methods allowing the targeted insertion of transgenes into a plant genome at desired loci by using homologous recombination combined with rare-cutting endonucleases without the need of inserting an exogenous selectable marker.

### BACKGROUND OF THE INVENTION

Genetic engineering of crop plants has traditionally involved the random insertion of a transgene into the plant's genome using methods such as Agrobacterium-mediated transformation or biolistic particles. Random insertion methods pose a number of potential drawbacks however. Firstly, expression of the transgene is often unpredictable due to its chromosomal environment and in many cases expression of the transgene is effectively silenced. Moreover, traditional transformation methods often lead to multiple copies of the transgene integrating into the genome which can cause difficulties in tracking multiple transgenes present on different chromosomes during segregation. Targeted insertion of transgenes at predetermined genomic loci would provide a solution to these problems, but in plant systems this has always been particularly difficult due to the very low rate of homologous recombination in plants.

Targeted genomic modification has been demonstrated in a number of eukaryotic systems including plants and has been achieved through several different methods to date. For example, insertion of a transgenic sequence into a eukaryotic organism can be achieved through homologous recombination by designing a DNA sequence flanked by sequences homologous to the genomic target (U.S. Pat. No. 5,527,695). In this case, screening of transformants relies on the inclusion of selectable marker within the engineered transgene construct. An improvement of homologous recombination methods involves the use of rare-cutting specific endonucleases such as engineered Zinc Finger Nucleases (ZFNs), enzymes which are engineered to create DNA double-strand breaks at specific loci and which can therefore be used to modify engineered reporter genes in plant systems (Lloyd et al 2005; Wright et al 2005). Such targeting systems also appear to increase the rate of localised homologous recombination. The use of ZFNs has been refined and shown to be a viable method for targeted mutagenesis in plant systems, to allow the alteration of desired genes through the precise modification of individual nucleotides (Townsend et al 2009).

The invention described hereunder provides methods which combine the targeted insertion of a transgene (knock-in) with the targeted mutagenesis of an endogenous selected gene, said transgene being inserted adjacent, preferably downstream, of the mutagenized gene. Accordingly, targeted mutagenesis is used to confer herbicide resistance to the plant cell, while the transgene is being inserted into the plant genome by homologous recombination, adjacent to said herbicide resistant gene, without requiring an exogenous selection marker.

The present invention is believed to be the first to show methods for producing a fertile plant having an altered genome comprising two or more site-specific insertions in a defined region of the genome of the plant.

### SUMMARY OF THE INVENTION

The present invention relates to improved methods for targeted insertion of transgenes at a single genetic locus in plant species. The invention makes use of a sequence-specific nuclease, preferably rare-cutting endonuclease, which is engineered to target an endogenous plant gene, such as acetolactate synthase (ALS), for which mutant versions of the gene are known to confer herbicide resistance, for instance to the herbicide chlorsulphuron. The invention provides methods for the preparation of a donor matrix, which is designed to introduce herbicide resistance mutations into said endogenous plant gene. Said donor matrix further comprises a transgene which is integrated at a site downstream of said endogenous coding region. Insertion of a transgene at a desired locus is thus achieved in tandem with modification of a native plant gene to confer herbicide resistance and therefore permits screening of putative transformants and elimination of transformants where transgene insertion has occurred randomly. The methods of the invention also permit subsequent insertion of transgenes at the same genetic locus, in particular through the inclusion of additional nuclease cleavage sites during the initial transformation, leading to efficient gene stacking.

### BRIEF DESCRIPTION OF THE DRAWINGS

FIG. 1: Strategy for coupling creation of herbicide resistance with targeted insertion of transgenes. Targeted gene modification is illustrated for the ALS1 and ALS2 loci of N. benthamiana. TALEN™ were engineered that cleave downstream of the ALS1 or ALS2 coding sequences. Cleavage stimulates homologous recombination between the chromosome and the donor DNA, which is illustrated above the chromosomes. Homologous recombination incorporates both a mutation in ALS, which confers herbicide resistance, and inserts the transgenes (gene stack) downstream of the coding sequences. In the example shown here, two different gene stacks are inserted downstream of each of the ALS1 and ALS2 genes.

FIG. 2: Donor matrices used for targeted insertion at the ALS loci in N. benthamiana. In all three donors, sequences that create a W568L mutation are included in the left homology arm. The donors differ by the sequences between the left and right homology arms. Located between the homology arms in the donor for the ALS1 knock-in is a 35S promoter that drives expression of a hygromycin phosphotrasferase gene (HPH) that is separated from the coding sequence for the yellow fluorescent protein (YFP) by a T2A translational skipping sequence. This same selectable/screenable marker cassette is used for one of the ALS2 knock-in donors. The other ALS2 knock-in donor only carries coding sequence for YFP. Nos-Ter refers to a transcriptional termination sequence from nopaline synthase.

FIG. 3: Examples of TALEN-induced mutations at ALS1 and ALS2. In the top line of each figure are the DNA sequences of the recognition sites for TALEN™ ALS1_T02 or ALS2_T02 (underlined and in capital letters). Below are shown representative mutations that were induced by imprecise NHEJ with the sizes of deletions given on the right.

### DETAILED DESCRIPTION OF THE INVENTION

According to a first embodiment of the invention, the method involves the use of TAL Effector Nucleases as sequence-specific endonucleases of choice to perform homologous recombination in plants. This type of endonuclease, which is further defined below, has shown to increase the efficiency of allelic replacement in plants and particularly targeted mutagenesis of the ALS gene. It is believed that TAL Effector Nucleases are particularly appropriate to perform targeting mutagenesis of endogenous plant genes as shown in the experimental part of this application.

Other types of sequence-specific nuclease (rare-cutting endonuclease) may be used to perform the invention as long as these are capable of inducing a double stranded DNA break precisely at one or more targeted genetic loci, resulting in one or more targeted mutations at that locus or loci and allowing the integration of a chosen transgene at a site up or downstream of the mutagenized region. Such sequence-specific nucleases include, but are not limited to, ZFNs (Zinc Finger Nucleases), engineered homing endonucleases such as I-SceI (WO9614408) and I-CreI (WO2004067736), MBBBDs (PCT/US2013/051783) and also Cas9/CRISPR systems (Jinek et al., 2012). Such sequence-specific nucleases are used in conjunction with a donor matrix, which generally further comprises left and right homologous arms to permit homologous recombination of the matrix at a targeted genomic location. In a further aspect of this embodiment the homologous arms contain one or more mutations to permit targeted mutation of a preselected genomic locus. In a further aspect of the invention, the donor matrix also comprises one or more transgenes to be inserted downstream of the site targeted by the sequence-specific nuclease. The donor matrix may also comprise one or more additional nuclease cleavage sites, which may allow for the later insertion of further transgene constructs at the same site. Such sites may include, but are not limited to, Cre-Lox recognition sites, sites for recognition by engineered or natural restriction endonucleases or meganucleases, like for instance I-SceI and I-CreI.

One or more mutations may be introduced by the method into the coding sequence of the gene to confer herbicide resistance. According to a preferred aspect of the invention, the mutation is introduced into a gene encoding ALS in order to confer resistance to chlorsulphuron. In a specific aspect of this embodiment, the mutation produces an amino acid substitution from W to L into the ALS protein, in particular into the W located at amino acid position 568 of the ALS protein encoded by the surB gene of Nicotiana tabacum (SEQ ID NO. 8). This position is highly conserved in many ALS proteins from various plant species, so that the invention can be applied to many of those plant species by identifying the corresponding position in proteins having identity to ALS. Most dicotyledonous species display ALS genes that are more than 75% identical to the tobacco surA and surB genes. The mutation edited in the gene may be any transition or transversion which confers herbicide resistance corresponding to said W to L substitution at position 568. Further mutations may be similarly or cumulatively generated into native gene sequences to confer herbicide resistance in view of obtaining transgene stacking. Such genes include but are not limited to PPO (protoporphyrinogen oxidase) and ESPS (3-phosphoshikimate 1-carboxyvinyltransferase). The invention also contemplates the situation where the inactivation of the gene by mutation induces a resistance to an herbicide such as, for example, the inactivation of genes encoding a polypeptide having nitrate reductase activity, which can confer plant cells resistance to chlorate.

One or more transgenes may be inserted by the matrix at a site adjacent to the endogenous gene, upstream or downstream of the mutagenized target, which produces a gene stack.

According to an aspect of the invention, an additional transgene may be introduced to encode a reporter gene or a selectable marker, although such reporter gene or selection marker is not necessary to carry out insertion of the transgene. Such additional transgenes include but are not limited to acetohydroxyacid synthase (AHAS), alkaline phosphatase (AP), beta galactosidase (LacZ), beta glucoronidase (GUS), chloramphenicol acetyltransferase (CAT), green fluorescent protein (GFP) and associated variants such as yellow fluorescent protein (YFP) and cyan fluorescent protein (CFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

In another embodiment of the invention, the inserted transgene is regulated under the control of the CaMV (Cauliflower Mosaic Virus) 35S constitutive promoter. In a further aspect, the inserted transgene can be regulated instead through a different constitutive promoter. Such constitutive promoters include, for example, the core promoter of the Rsyn7 promoter and other constitutive promoters disclosed in PCT Publication No. WO 99/43838 and U.S. Pat. No. 6,072,050. In a further aspect of this embodiment, the promoter may be an inducible promoter, such as a chemically induced promoter. Chemically regulated promoters can be used to modulate the expression of a gene in a plant through the application of an exogenous chemical regulator. Depending upon the objective, the promoter may be a chemical inducible promoter, where application of the chemical induces gene expression, or a chemical repressible promoter, where application of the chemical represses gene expression. Chemical inducible promoters are known in the art and include, but are not limited to, the maize 1n2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemically regulated promoters of interest include steroid-responsive promoters (see, for example, the glucocorticoid-inducible promoter in Schena et al. (1991) Proc. Natl. Acad. Sci. USA 88:10421-10425 and tetracycline-inducible and tetracycline-repressible promoters (see, for example, U.S. Pat. Nos. 5,814,618 and 5,789,156. Tissue-preferred promoters can be utilized to permit expression within a particular plant tissue. Tissue-preferred promoters include Yamamoto et al. (1997) Plant J. 12(2):255-265; Kawamata et al. (1997) Plant Cell Physiol. 38(7):792-803. Such tissue-specific promoters may also include root-preferred promoters which can be selected from the many available from the literature or isolated de novo from various compatible species. See, for example, Hirel et al. (1992) Plant Mol. Biol. 20(2): 207-218 (soybean root-preferred glutamine synthetase gene). Seed-specific promoters (those promoters active during seed development such as promoters of seed storage proteins) as well as “seed-germinating” promoters (those promoters active during seed germination) are also known. See Thompson et al. (1989) BioEssays 10:108. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529.

The method of the invention may apply to any plant species, insofar as they contain an endogenous gene that can confer herbicide resistance upon mutagenesis. Such plants may be any monocot or dicot plant, such as but not limited to Arabidopsis; field crops (e.g., alfalfa, barley, bean, corn, cotton, flax, pea, rape, rice, rye, safflower, sorghum, soybean, sunflower, tobacco, and wheat); vegetable crops (e.g., asparagus, beet, broccoli, cabbage, carrot, cauliflower, celery, cucumber, eggplant, lettuce, onion, pepper, potato, pumpkin, radish, spinach, squash, taro, tomato, and zucchini); fruit and nut crops (e.g., almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber). In a preferred embodiment of the invention, the plant species used is Nicotiana sp., more preferably N. benthamiana.

In another embodiment of the invention, the donor matrix is encoded for by a plasmid vector to allow transfection of a suitable plant species. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. An expression vector may comprise, but is not limited to, a YAC (yeast artificial chromosome), a BAC (bacterial artificial), a baculovirus vector, a phage, a phagemid, a cosmid, a viral vector, a plasmid, a RNA vector or a linear or circular DNA or RNA molecule which may consist of a chromosomal, non chromosomal, semi-synthetic or synthetic DNA. In general, expression vectors of utility in recombinant DNA techniques are often in the form of “plasmids” which refer generally to circular double stranded DNA loops which, in their vector form are not bound to the chromosome. Large numbers of suitable vectors are known to those of skill in the art and commercially available, such as the following bacterial vectors: pQE70, pQE60. pQE-9 (Qiagen), pbs, pD10, phagescript, psiXI74. pbluescript SK. pbsks. pNH8A. pNH16A, pNH18A, pNH46A (Stratagene); ptrc99a, pKK223-3, pKK233-3, pDR540, pRIT5 (Pharmacia); pWLNEO. pSV2CAT, pOG44, pXT1, pSG (Stratagene); pSVK3, pBPV, pMSG, pSVL (Pharmacia); pQE-30 (QIAexpress).

In another embodiment of the invention the plasmid encoding the donor matrix is inserted into the plant genome via PEG-mediated transformation of isolated protoplasts. In a further aspect of this embodiment the plasmid may be inserted via electroporation or through biolistic transformation methods or through any other suitable transfection method. Such methods for introducing an expression vector into a plant are known in the art. In the case of biolistic transformation, the expression vector is introduced into plant tissues with a biolistic device that accelerates the microprojectiles to speeds of 300 to 600 m/s which is sufficient to penetrate plant cell walls and membranes (See Klein et al., 1992).

Another method for introducing DNA to plants is via the sonication of target cells. Alternatively, liposome or spheroplast fusion has been used to introduce expression vectors into plants (see e.g. Christou et al., 1987). Direct uptake of DNA into protoplasts using CaCl2 precipitation, polyvinyl alcohol or poly-L-ornithine has also been reported (see e.g. Draper et al., 1982). Electroporation of protoplasts and whole cells and tissues has also been described (Laursen et al., 1994).

### DEFINITIONS

As used herein the term “ALS” refers to “Acetolactate synthase” also known as acetohydroxy acid synthase, or AHAS, said enzyme catalysing the first step in the synthesis of branched chain amino acids. For instance, two ALS genes can be found in N. tabacum, surA and surB, the gene sequences of which are respectively referred to under SEQ ID NO.7 and SEQ ID NO.8. Accession numbers for these genes in unified database are respectively X07644 and X07645. This term also applies to any homologous native plant protein having similar function or identity with acetolactate synthase. Such homologous ALS genes are found in various plant species, as for instance Solanum tuberosum (Potato) see SEQ ID NO.9, Capsicum annum (Sweet Pepper) SEQ ID NO.10.

By “W568L” mutation is more particularly meant a mutation from W (Tryptophan) to L (Lysine) at position 568 in the SurA and/or SurB protein from Nicotiana tabacum encoded by surA and sure genes (SEQ ID NO.7 and NO.8). This particular mutation results in a form of acetolactate synthase that is resistant to the herbicide chlorsulfuron. However, the W that is altered to confer herbicide resistance is highly conserved among plant ALS proteins. For instance, in Nicotiana benthamiana, the corresponding mutation is W570L. Corresponding positions can be easily identified by performing BLAST alignments among ALS proteins showing identity with SurA and SurB and introduced according to the invention into the plant species containing the genes encoding these proteins. Other examples of mutations in ALS protein conferring herbicide resistance, in particular to sulfonylurea and imidazolinone herbicides, are “P191A” (with respect to the protein encoded by SEQ ID NO.8), which has at least a corresponding mutation P193A into ALS2 of N. benthamiana, and also “S647T” (with respect to the protein encoded by SEQ ID NO.8) and its corresponding mutation S649T into ALS2 of N. benthamiana. Proline and serine positions corresponding to P191 and S647 are easily identified into homologous ALS protein having identity with SurB of N. tabacum, because these positions are generally highly conserved when aligning these proteins using BLASTP.

As used herein the term “Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. BLASTP may also be used to identify an amino acid sequence having at least 80%, 85%, 87.5%, 90%, 92.5%, 95%, 97.5%, 98%, 99% sequence similarity to a reference amino acid sequence using a similarity matrix such as BLOSUM45, BLOSUM62 or BLOSUM80. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The same applies with respect to polynucleotide sequences using BLASTN.

As used herein the term “endonuclease” refers to an enzyme capable of causing a double-stranded break in a DNA molecule.

As used herein the term “sequence-specific nuclease” refers to any nuclease enzyme which is able to induce a double-strand DNA break at a desired and predetermined genomic locus

As used herein the terms “rare-cutting endonuclease” and “meganuclease” refer to natural or engineered sequence-specific nuclease, typically having a polynucleotide recognition site of about 10 to 40 bp in length, more preferably of 14 to 40 bp. Typical meganucleases are homing endonucleases, more particularly belonging to the dodecapeptide LAGLIDADG family (WO 2004/067736), which can cause cleavage inside their recognition site, leaving 4 nt staggered cut with 3′OH or 5′OH overhangs. As used herein the term “homing endonuclease” designates double stranded DNAses that have large, asymmetric recognition sites (12-40 base pairs). Examples include I-Sce I, I-Chu I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, PI-Tsp I and I-Msol. Other rare-cutting endonucleases, more particularly referred to in this application, are chimeric endonucleases made of a fusion of an engineered binding domain specific to a polynucleotide sequence with an endonuclease catalytic domain. Such chimeric endonucleases can be represented by zinc-finger-nucleases (ZFN), TAL-effector endonucleases or any nuclease fused to modular base-per-base binding domains (MBBBDs) as referred to in PCT/US2013/051783—Such chimeric endonucleases are able to bind a predetermined nucleic acid target sequence and induce cleavage in said sequence or a sequence adjacent thereto.

As used herein the term “zinc finger nuclease” (ZFN) refers to artificial restriction enzymes generated by fusing a zinc finger DNA-binding domain to a DNA-cleavage domain. Briefly, ZFNs are synthetic proteins comprising an engineered zinc finger DNA-binding domain fused to the cleavage domain of an endonuclease, such as Fok1. ZFNs may be used to induce double-stranded breaks in specific DNA sequences and thereby promote site-specific homologous recombination and targeted manipulation of genomic sequences.

As used herein the term “TAL-effector endonuclease” refers to artificial restriction enzymes generated by fusing a DNA recognition domain deriving from TALE proteins of Xanthomonas to a catalytic domain of a nuclease, for instance FokI and I-TevI, as respectively described in WO 2011/072246 and WO 2012/138927. TAL-effector endonuclease can be referred to herein as TALEN™, which is trade mark owned by Cellectis (Cellectis SA, 8, rue de la Croix Jarry, 75013 PARIS).

Methods for selecting endogenous target sequences and generating TALEN™ targeted to such sequences can be performed as described elsewhere. See, for example, PCT Publication No. WO 2011/072246, which is incorporated herein by reference in its entirety. Transcription activator-like (TAL) effectors are found in plant pathogenic bacteria in the genus Xanthomonas. These proteins play important roles in disease, or trigger defense, by binding host DNA and activating effector-specific host genes (see, e.g., Gu et al., Nature 435:1122-1125, 2005; Yang et al., Proc. Natl. Acad. Sci. USA 103:10503-10508, 2006; Kay et al. Science 318:648-651, 2007; Sugio et al., Proc. Natl. Acad. Sci. USA 104:10720-10725, 2007; and Römer et al. Science 318:645-648, 2007). Specificity depends on an effector-variable number of imperfect, typically 34 amino acid repeats (Schornack et al., J. Plant Physiol. 163:256-272, 2006). Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. This mechanism for protein-DNA recognition enables target site prediction for new target specific TAL effectors, as well as target site selection and engineering of new TAL effectors with binding specificity for the selected sites. TAL effector DNA binding domains can be fused to other sequences, such as endonuclease sequences, resulting in chimeric endonucleases targeted to specific, selected DNA sequences, and leading to subsequent cutting of the DNA at or near the targeted sequences. Such cuts (i.e., double-stranded breaks) in DNA can induce mutations into the wild type DNA sequence via NHEJ or homologous recombination, for example. In some cases, TALEN™ can be used to facilitate site directed mutagenesis in complex genomes, knocking out or otherwise altering gene function with great precision and high efficiency. As described in the examples herein, TALENs can be used to mutagenize the endogenous genes, thereby promoting site-specific homologous recombination.

As used herein the term “modular base-per-base binding domains” (MBBBDs) designate engineered binding domain using the assembly of new modular polypeptides having specificity to nucleic acid bases, which originate more particularly from the microorganism Burkholderia rhizoxinica (PCT/US2013/051783). These engineered modular binding domains can be used as an alternative of the above TALE binding domains derived from Xanthomonas in fusion, for instance, with Fok1 and I-Tev1 nuclease domains.

Another type of rare-cutting endonuclease is referred to herein as “Cas9/CRISPR system”. This system is characterized by the combined use of an endonuclease from the bacterial Cas9 family and of a single stranded guide RNA that guides said endonuclease to a DNA target sequence generally of 20 base pairs. This DNA target is generally chosen to be located in the genome upstream so-called PAM (protospacer adjacent motif) sequence motives (NGG or NAG) recognized by Cas9. The guide RNA molecule (gRNA), which is generally a single stranded RNA is introduced into the living cell to confer cleavage and specificity to Cas9. It is a synthetic RNA designed to match the desired 20 bp sequence in the genome upstream the PAM. The use of Cas9/CRISPR in plants has been reviewed by Belhaj et al. (2013), which is incorporated by reference.

As used herein the term “mutagenesis” refers to processes in which mutations are introduced into a selected DNA sequence. In the methods described herein, for example, mutagenesis occurs via a double stranded DNA breaks made by TALEN™ targeted to selected DNA sequences in a plant cell. Such mutagenesis results in “TALEN-induced mutations”, which can modify, reduce of unable expression of the targeted gene. Following mutagenesis, plants can be regenerated from the treated cells using known techniques (e.g., planting seeds in accordance with conventional growing procedures, followed by self-pollination). In the sense of the present invention, mutagenesis is not limited to punctual mutations. Any gene repair or deletion performed on the endogenous gene (promoter of coding sequence) conferring herbicide resistance to the plant is regarded as a mutation of the gene.

As used herein the term “homologous” is intended a sequence with enough identity to another one to lead to a homologous recombination between sequences, more particularly having at least 95% identity, preferably 97% identity and more preferably 99%.

As used herein, the term “adjacent” means downstream or upstream of a genetic locus. In the context of the present invention, the heterologous gene can be inserted with the donor matrix so as to introduce a genetic modification in the gene conferring herbicide resistance upstream or downstream said herbicide resistance gene, which means that the insertion of the heterologous gene will not prevent the expression of the resistance gene, but will be in sufficient proximity of said gene to be brought on the same donor matrix. Generally adjacent means less than 20 kb from the herbicide resistance gene, preferably less than 10 kb, more preferably less than 5 kb, even more preferably less than 1 kb.

As used herein the term “herbicide” designates any chemical substance that inhibits the growth of the plant. The resistance by a plant to an herbicide may be partial, for instance when this resistance occurs with respect to a certain concentration of the substance, in presence/absence of co-factors, or external factors like temperature, humidity etc.

As used herein the term “vector” designates any nucleic acid construct used to cell transfection: viral vector, plasmid, RNA vector or a linear or circular DNA or RNA molecule, which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. This term can also be used in the present invention, for instance, to designate a donor matrix—i.e. a nucleic acid construct carrying the sequences homologous to that of the endogenous gene and the transgene sequence to be inserted according to the method of the present invention.

More specifically, the present invention is more particularly drawn to the following embodiments:

1. A method for targeted genetic insertion into a plant genome, preferably without inserting an exogenous selectable marker into said genome, said method comprising one of several of the following steps:

a) Providing a plant cell which comprises an endogenous gene that can be modified to confer herbicide resistance, for instance ALS (acetolactate synthase), PPO (protoporphyrinogen oxidase), ESPS (3-phosphoshikimate 1-carboxyvinyltransferase), nitrate reductase, or a homologous gene thereof.

b) Obtaining a donor matrix comprising a sequence homologous to said endogenous gene, said homologous sequence introducing a genetic modification to render said gene capable of conferring herbicide resistance to the cell, and adjacent (downstream or upstream) of said homologous sequence, a desired transgene to be inserted into the genome;

c) Transformation of the plant with said donor matrix;

d) Further transforming said plant cell with a nucleic acid expressing a sequence-specific nuclease, preferably a rare-cutting endonuclease, to specifically cleave said gene susceptible to confer herbicide resistance;

e) Expressing said sequence-specific nuclease into said cell in order to induce homologous recombination between the endogenous gene and the donor matrix;

to produce a plant cell having resistance to herbicide, in which stable integration of the transgene has occurred downstream of the endogenous gene conferring said resistance.
Said method may comprise an additional step where the plant cell is grown using the herbicide the modified gene confers resistance to.
2. The method as above, wherein the sequence-specific nuclease is a rare-cutting endonuclease.
3. The method as above, wherein rare-cutting endonuclease is a meganuclease, a chimeric endonuclease or a Cas9/CRISPR system.
4. The method as above, wherein the rare-cutting endonuclease is a TAL-Effector endonuclease.
5. The method as above, wherein the meganuclease is a homing endonuclease.
6. The method as above, wherein the rare-cutting endonuclease is a Zinc Finger Nuclease.
7. The method as above, wherein the endogenous plant gene expresses ALS (acetolactate synthase).
8. The method as above, wherein the endogenous plant gene expresses a polypeptide having nitrate reductase activity, and wherein said nitrate reductase activity is inactivated by introduction of said mutation in step b).
9. The method as above, wherein said mutation confers resistance to chlorate.
10. The method as above, wherein the endogenous plant gene has at least 75%, preferably at least 80%, more preferably at least 90%, even more preferably at least 95% identity with SEQ ID NO. 7 or SEQ ID NO.8.
11. The method as above, wherein said sequence homologous to said endogenous gene comprised on said matrix allows the expression of a functional ALS protein by the cell after homologous recombination.
12. The method as above, wherein said functional ALS protein has a mutation corresponding to P191A, W568L, or S647T.
13. The method as above, wherein the cell in which the transgene is inserted is selected on the resistance to herbicide conferred by the modified endogenous gene.
14. The method as above, wherein said herbicide is sulfonylurea, such as chlorsulfuron, or imidazolinone herbicides.
15. The method as above, wherein at least two endogenous genes are selected for transgene insertions.
16. The method as above, wherein at least two genes having identity with ALS genes are used for transgene insertions.
17. The method as above, wherein said two genes are respectively ALS1 and ALS2.
18. The method as above, wherein expression of the transgene is regulated by a constitutive promoter, such as the Cauliflower Mosaic Virus 35S promoter.
19. The method as above, wherein the expression of the transgene is regulated by an inducible promoter, such as the steroid-inducible glucocorticoid responsive promoter.
20. The method as above, wherein the expression of the transgene is regulated by a tissue specific promoter.
21. The method as above, wherein the transgene encodes for a therapeutic protein, such as a vaccine.
22. The method as above, wherein said donor matrix comprises a pair of left and right arms, said arms having homology to the genetic locus to be targeted.
23. The method as above, wherein at least one arm contains at least one engineered mutation to permit mutation of the endogenous plant gene by homologous recombination.
24. The method as above, wherein said donor matrix comprises one or more additional nuclease cleavage sites for the insertion of one or more additional transgenes subsequent to the initial plant transformation
25. The method as above, wherein said donor matrix is encoded by a plasmid vector
26. The method as above, wherein said donor matrix is encoded by an episomal vector
27. The method as above, wherein said plant species is a field crop, such as but not limited to alfalfa, barley, bean, corn, cotton, flax, pea, rape, rice, rye, safflower, sorghum, soybean, sunflower, tobacco, wheat.
28. The method as above, wherein said plant genus is Nicotiana and the species preferably N. benthamiana.
29. The method as above, wherein said plant species is a vegetable crop, such as but not limited to asparagus, beet, broccoli, cabbage, carrot, cauliflower, celery, cucumber, eggplant, lettuce, onion, pepper, potato, pumpkin, radish, spinach, squash, taro, tomato, and zucchini.
30. The method as above, wherein said plant species is a fruit crop, such as but not limited to almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon.
31. The method as above, wherein said plant species is an ornamental, such as but not limited to alder, ash, aspen, azalea, birch, boxwood, camellia, carnation, chrysanthemum, elm, fir, ivy, jasmine, juniper, oak, palm, poplar, pine, redwood, rhododendron, rose, and rubber.
32. The method as above, wherein transformation is effected through insertion of the donor matrix construct into isolated plant protoplasts.
33. The method as above, wherein transformation is effected through insertion of the donor matrix construct into isolated plant protoplasts through PEG (polyethylene glycol) mediated transfection.
34. The method as above, wherein transformation is effected through insertion of the donor matrix construct into isolated plant protoplasts through electroporation.
35. The method as above, wherein transformation is effected through insertion of the donor matrix construct into isolated plant protoplasts through biolistic mediated transfection.
36. The method as above, wherein transformation is effected through insertion of the donor matrix construct into isolated plant protoplasts sonication mediated transfection.
37. The method as above, wherein transformation is effected through insertion of the donor matrix construct into isolated plant protoplasts through liposome mediated transfection.
38. The method as above, wherein transformation is effected through insertion of the donor matrix construct into isolated plant protoplasts through direct DNA uptake transfection, such as but not limited to CaCl₂ uptake transfection.
39. A transformed plant cell obtainable according to the above method.
40. A herbicide resistant plant grown or cultured from the above plant cell, a seed thereof, or progeny thereof having herbicide resistance.
41. A transformed plant cell having a transgene in its genome, preferably two transgenes, respectively inserted adjacent to at least one gene having at least 75%, preferably at least 80%, more preferably at least 90%, even more preferably at least 95% identity with ALS genes, more particularly SEQ ID NO. 7 or 8.
42. A transformed plant cell according to the invention, wherein at least one of its ALS protein displays mutations corresponding to P191A, W568L, or S647T.
43. A transformed plant cell according to the invention, wherein said plant is resistant to sulfonylurea or imidazolinone herbicides.
44. A transformed plant cell according to the invention, wherein said plant cell is resistant to chlorsulfuron.
45. A transformed plant cell according to the invention, wherein said plant cell does not comprise any further transgenes in its genome.
46. A transformed plant cell according to the invention, wherein said transgene does not comprise any exogenous selection marker.
47. A kit for the targeted genetic modification of a plant species comprising a donor matrix as previously defined and a vector encoding a meganuclease designed to target an endogenous gene involved into herbicide resistance, and optionally, plant cells having an endogenous gene that can be modified to confer herbicide resistance, reagents, supplies, or equipment for transforming a plant cell, separate containers for each ingredient, packaging materials, and/or instructions for use in preparing a herbicide-resistant plant cell.
48. A vector containing a donor matrix comprising a sequence homologous to an endogenous plant cell gene, said homologous sequence including a genetic modification to render the endogenous plant cell gene capable of conferring herbicide resistance to the cell, and downstream of said homologous sequence, a desired transgene to be inserted into the genome, and optionally, a gene encoding a sequence specific nuclease to specifically cleave said endogenous plant cell gene.
49. A host cell comprising a vector containing a donor matrix comprising a sequence homologous to an endogenous plant cell gene, said homologous sequence including a genetic modification to render said gene capable of conferring herbicide resistance to the cell, and downstream of said homologous sequence, a desired transgene to be inserted into the genome and optionally a gene encoding a sequence specific nuclease to specifically cleave said endogenous plant cell gene.
The following examples further illustrate the invention without intending to limit its scope.

### EXAMPLES

### Example 1

### Engineering Sequence-Specific Nucleases that Target the N. benthamiana ALS Gene

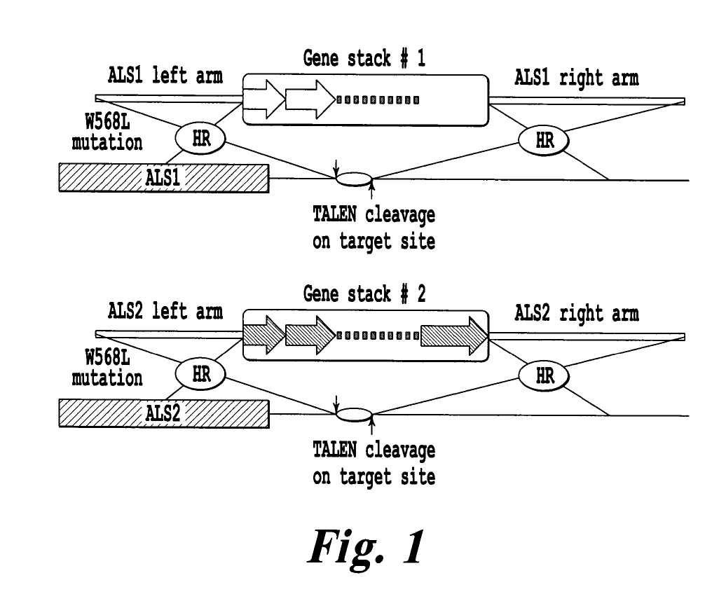

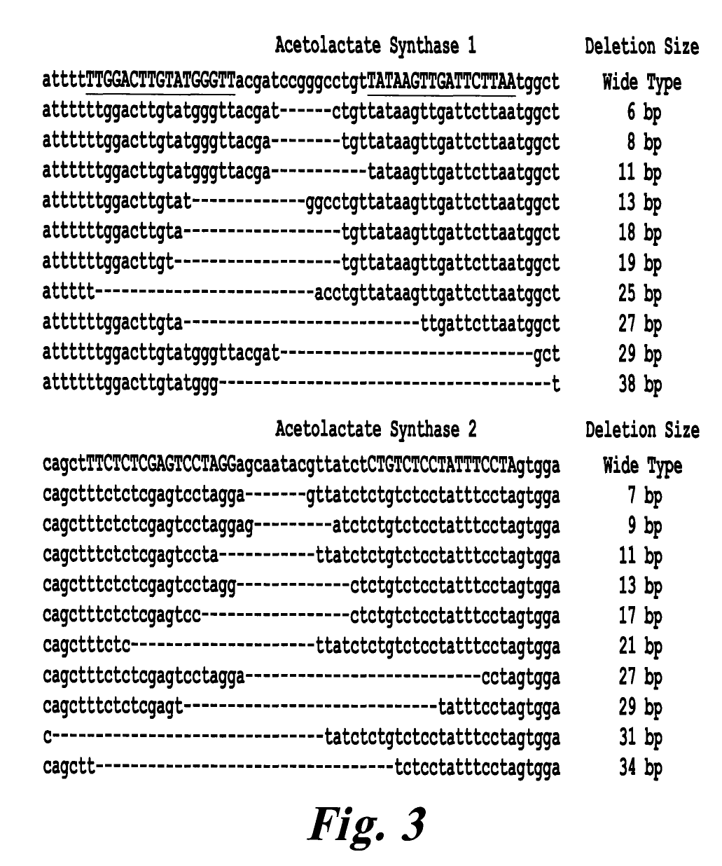

N. benthamiana encodes two ALS genes designated ALS1 and ALS2. To stimulate homologous recombination in either or both of the N. benthamiana ALS genes, sequence-specific nucleases were designed that target sites just downstream of the protein coding sequence (FIGS. 1 and 3). Although different sequence-specific nucleases could be used to create a targeted double strand break in ALS, transcription activator-like effector nucleases (TALEN™) were chosen (Christian et al. 2010). For ALS1, two TALEN™ pairs were designed to target two different sites downstream of ALS using software that specifically identifies TALEN™ recognition sites, such as TALE-NT 2.0 (Doyle et al 2012) (SEQ ID NO. 1-4). These TALEN™ are designated ALS1_T01 and ALS1_T02. Two TALEN™ were also engineered to target two sites downstream of ALS2 and are designated ALS2_T01 and ALS2_T02 (SEQ ID NO.1-4). TALEN™ were synthesized by Cellectis (8, rue de la Croix Jarry, 75013 PARIS) using a method as described in WO2013017950.

### Example 2

### Activity in Yeast of TALENs Targeting ALS

To assess the activity of the TALEN™ targeting the N. benthamiana ALS loci, activity assays were first performed in yeast by methods similar to those previously described (Christian et al. 2010). For these assays, a target plasmid was constructed with the TALEN™ recognition site cloned in a non-functional β-galactosidase reporter gene. The target site is flanked by a direct repeat of β-galactosidase coding sequence such that if the reporter gene is cleaved by the TALEN, recombination occurs between the direct repeats and restores function to the β-galactosidase gene. B-galactosidase activity, therefore, served as a measure of TALEN™ cleavage activity.

The activity of the ALS TALEN™ pairs was tested in yeast, and all four showed high cleavage activity under two distinct temperature conditions (i.e. 37° C. and 30° C.). Cleavage activities were normalized to the benchmark nuclease, I-SceI, and the results are summarized in Table 1. [* Normalized to I-SceI (max=1.0)]

TABLE 1
ALS1 and ALS2 TALENT ™ Activity in Yeast.

| | | Activity | |
|---|---|---|---|
| | | in yeast* | |
| Name | TALEN target | 37° C. | 30° C. |
| ALS1_T01 | TTTAGTGCGATAAAGTT | 0.93 | 0.83 |
| | AGCTTGTTTCCACATTT | | |
| | TTATTTCGTAAGCTA | | |
| ALS1_T02 | TTGGACTTGTATGGGTT | 0.95 | 0.90 |
| | ACGATCCGGGCCTGTTA | | |
| | TAAGTTGATTCTTAA | | |
| ALS2_T01 | TAGCTTGTTCCACATTT | 1.0 | 0.9 |
| | TTATTTCATAAGCTATG | | |
| | TCATGCTGGGTCAGA | | |
| ALS2_T02 | TTCTCTCGAGTCCTAGG | 1.0 | 0.9 |
| | AGCAATACGTTATCTCT | | |
| | GTCTCCTATTTCCTA | | |

### Example 3

### Activity of the ALS TALEN™ at their Endogenous Target Sites in N. benthamiana

TALEN activity at endogenous target sites in N. benthamiana was measured by expressing the TALENs in protoplasts and surveying the TALEN™ target sites for mutations introduced by non-homologous end-joining (NHEJ). Methods for protoplast preparation were as previously described (Wright et al. 2005). Briefly, seeds were sterilized by washing them successively on 100% ethanol, 50% bleach and then sterile distilled water. The sterilized seeds were planted on MS agarose medium supplemented with iron. Protoplasts were isolated from young expanded leaves using the protocol described by Wright et al, 2005.

TALEN-encoding plasmids together with a YFP-encoding plasmid were next introduced into N. benthamiana protoplasts by PEG-mediated transformation (Yoo et al 2007). Twenty-four hours after treatment, transformation efficiency was measured by evaluating an aliquot of the transformed protoplasts using a flow cytometer to monitor YFP fluorescence. The remainder of the transformed protoplasts was harvested, and genomic DNA was prepared by a CTAB-based method. Using the genomic DNA prepared from the protoplasts as a template, an approximately 300-bp fragment encompassing the TALEN™ recognition site was amplified by PCR. The PCR product was then subjected to 454 pyro-sequencing. Sequencing reads with insertion/deletion (indels) mutations in the spacer region were considered as having been derived from imprecise repair of a cleaved TALEN™ recognition site by non-homologous end-joining (NHEJ). Mutagenesis frequency was calculated as the number of sequencing reads with NHEJ mutations out of the total sequencing reads. The values were then normalized by the transformation efficiency. The activity of the TALEN™ pairs is summarized in Table 2. Both TALEN™ pairs for ALS2 induced very high frequencies of NHEJ-induced mutations, ranging from 66% to 74%. One of the ALS1 TALEN™, namely ALS1_T02, induced mutagenesis at a frequency approximating 5%. The ALS1_T01 TALEN™ did not show activity above the negative control. Examples of TALEN-induced mutations in the ALS locus are shown in FIG. 3.

TABLE 2
454 Pyro-Sequencing Data for ALS1 and ALS2 TALEN ™

| | Location of | NHEJ mutagenesis freq | NHEJ mutagenesis freq. | Protoplast transformation |
|---|---|---|---|---|
| TALEN name | target site | with TALEN* | with negative control** | Efficiency |
| ALS1_T01 | ALS1 | 0.43% (3809) | 0.55% | 85% |
| ALS1_T02 | ALS1 | 4.9% (2950) | 0.55% | 81% |
| ALS2_T01 | ALS2 | 74.0% (6385) | 0.25% | 84% |
| ALS2_T02 | ALS2 | 66.9% (9092) | 0.10% | 85% |

*NHEJ mutagenesis frequency was obtained by normalizing the percentage of 454 reads with NHEJ mutations to the protoplast transformation efficiency. The total number of 454 sequencing reads used for this analysis is indicated in parentheses.
**Negative controls were obtained from protoplasts transformed only by the YFP-coding plasmid.

### Example 4

### Creating a Donor Matrix for Modifying the N. benthamiana ALS Locus

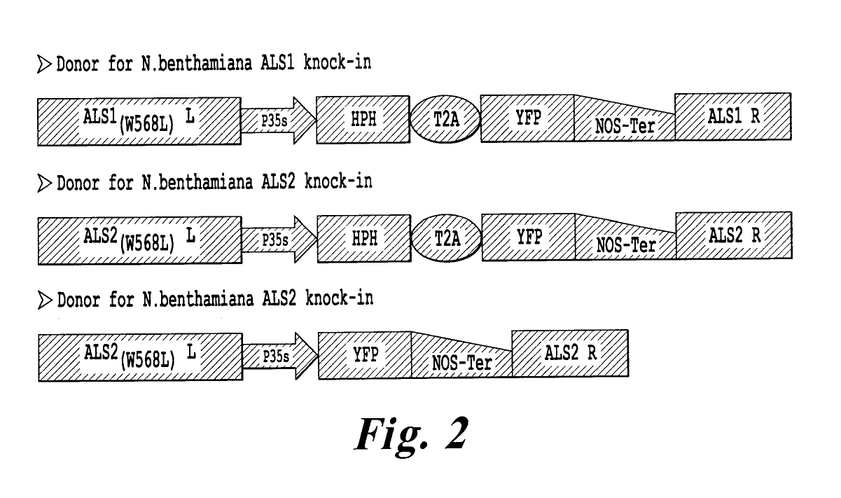

Recombination donor matrices were made to incorporate specific DNA sequence modifications into the ALS loci. As illustrated in FIG. 2, these matrices have two flanking ALS-specific homology arms (designated ALS1 L and ALS1 R or ALS2 L and ALS2 R). Incorporated in the ALS L homology arms for both genes were sequence modifications that introduce the W568L mutation that confers herbicide resistance. Between the homology arms were coding sequences for different marker gene cassettes that confer selectable or screenable phenotypes. One such marker cassette encoded a selectable marker, namely hygromycin phosphotransferase (HPH), followed by a screenable marker, namely the yellow fluorescent protein (YFP). The coding sequences for HPH and YFP were preceded by a 35S promoter and followed by a nopaline synthase (NOS) terminator. The two coding sequences were separated by a viral T2A translational skipping sequence that allows translation of both proteins from a single mRNA. A separate marker cassette designed for ALS2 only encoded YFP. The DNA sequences for the ALS1 and ALS2 donor matrices are provided in SEQ ID NO. 5, SEQ ID NO. 6, and SEQ ID NO. 11, respectively.

### Example 5

### Creating Plants with Targeted Insertions at ALS

Based on the 454 pyro-sequencing data, the TALEN™ pairs with the highest cleavage activity targeting each gene (i.e. ALS1_T02 and ALS2_T01) were chosen to create tobacco plants with targeted insertions downstream of the ALS coding sequences. Protoplasts were isolated from sterile tobacco leaves, and transformed with plasmids encoding TALEN™ targeting one of the loci and the corresponding donor matrix. After transformation, protoplast-derived calli were generated and selected for resistance to chlorsulfuron and/or hygromycin resistance as previously described (Van den Elzen et al. 1985; Townsend et al. 2009). Resistant calli could also be scored for YFP fluorescence by light microscopy. DNA was prepared from calli that were resistant and expressed YFP. The DNA was analyzed by PCR to assess whether the observed phenotypes were due to modification of the ALS gene and insertion of the markers using specific primers (SEQ ID NO. 12 and SEQ ID NO. 13).

The TALEN-mediated targeted insertion efficiency is summarized in Table 3. As described above, after delivery of TALEN™ and relevant donor matrices to protoplasts, calli were selected that were resistant to hygromycin or chlorsulfuron and screened for targeted insertion by PCR. Targeted insertions were recovered at both ALS1 and ALS2 at high frequencies of 15.34% and 12.34% respectively. In calli derived from protoplasts transformed with donor matrices alone (i.e. without TALEN™), targeted insertions were also observed, but at lower frequencies (5.61% for ALS1 and 1.15% for ALS2). A control transformation group was evaluated that was transformed with both TALENs and a donor matrix, however, no chlorsulfuron selection was applied. After genotyping 1,200 calli by PCR, no targeted insertions were identified in this control group. This indicates that the chlorsulfuron tolerance enabled by the W568L mutation was critical for enrichment of targeted insertion events.

Candidate calli with targeted insertions were regenerated into whole plants by first transferring them to shoot-inducing medium. After shoots of a few cm in length emerged, they were cut at the base and transferred to root-inducing medium. Once roots formed, they were transferred to soil. Targeted modification of the ALS locus is confirmed by additional PCR analyses, Southern blotting and DNA sequencing of the recombinant ALS loci. Seeds are collected from the modified plants and inheritance of the trait is monitored in the progeny to confirm stable, heritable transmission of the modified loci.

TABLE 3
Summary of data demonstrating TALEN ™ mediated targeted insertion.

| | | # of | # of targeted | % of targeted |
|---|---|---|---|---|
| Treatment | Selection | events | insertions | insertions |
| ALS1 TALEN + ALS1 donor | Hygromycin | 313 | 48 | 15.34% |
| (SEQ ID NO. 5) | | | | |
| ALS1 donor | Hygromycin | 107 | 6 | 5.61% |
| (SEQ ID NO. 5) | | | | |
| ALS2 TALEN + ALS2 donor | Chlorsulfuron | 316 | 39 | 12.34% |
| (SEQ ID NO. 11) | | | | |
| ALS2 donor | Chlorsulfuron | 262 | 3 | 1.15% |
| (SEQ ID NO. 11) | | | | |
| ALS2 TALEN + ALS2 donor | N/A | 1200 | 0 | 0.00% |
| (SEQ ID NO. 11) | | | | |

### SEQUENCE LISTING

The following sequences are the target sequences for the ALS1 and ALS2 TALEN™ used in the examples. The DNA sequences depicted are located downstream of the ALS1 or ALS2 coding sequences. Two TALEN™ pairs were designed for each gene and the underlined sequences represent the TALEN™ recognition sites:

SEQ ID NO. 1: ALS1_T01
GATTAATTTCTAGTGGAGTAGTTTAGTGCGATAAAGTTAGCTTGTTT
CCACATTTTTATTTCGTAAGCTATGTCAGCCAGGGTCAGATTGGAAC
TAAAGGTGTTAAATGGGTGGGTCGGGCCGGGCTTCTATTTTTTGGAC
TTGTATGGGTTACGATCCGGGCCTGTTATAAGTTGATTCTTAATGGC
TTCGGGTTCATCCGGGTAAAAATTGAACCATAAGGGTTACTGGTTGA
GGGGGCCGGATCGTGCCGGGTTTA
SEQ ID NO. 2: ALS1_T02
GATTAATTTCTAGTGGAGTAGTTTAGTGCGATAAAGTTAGCTTGTTT
CCACATTTTTATTTCGTAAGCTATGTCAGCCAGGGTCAGATTGGAAC
TAAAGGTGTTAAATGGGTGGGTCGGGCCGGGCTTCTATTTTTTGGAC
TTGTATGGGTTACGATCCGGGCCTGTTATAAGTTGATTCTTAATGGC
TTCGGGTTCATCCGGGTAAAAATTGAACCATAAGGGTTACTGGTTGA
GGGGGCCGGATCGTGCCGGGTTTA
SEQ ID 3: ALS2_T01
GATTAATTTCTAATGGAGTAGTTTAGTGTAATAAAGTTAGCTTGTTC
CACATTTTTATTTCATAAGCTATGTCATGCTGGGTCAGATTGGAACT
TCCTCTTTAGGTTGGATGTAATCCCTATTAGGGCTTTCTCTTAATTT
TATTATTGAATTGTTGGCTTTTAATCTGAGCAAGTTGATTTGCAGCT
TTCTCTCGAGTCCTAGGAGCAATACGTTATCTCTGTCTCCTATTTCC
TAGTGGATAATCTTATGATGGAAATATGT
SEQ ID NO. 4: ALS2_T02
GATTAATTTCTAATGGAGTAGTTTAGTGTAATAAAGTTAGCTTGTTC
CACATTTTTATTTCATAAGCTATGTCATGCTGGGTCAGATTGGAACT
TCCTCTTTAGGTTGGATGTAATCCCTATTAGGGCTTTCTCTTAATTT
TATTATTGAATTGTTGGCTTTTAATCTGAGCAAGTTGATTTGCAGCT
TTCTCTCGAGTCCTAGGAGCAATACGTTATCTCTGTCTCCTATTTCC
TAGTGGATAATCTTATGATGGAAATATGT
SEQ ID NO. 5: Donor sequence for ALS1 knock-in
(HPH and YFP; the underlined sequences represent
the homology arms for homologous recombination)
GAATTCACTATTGGAAAGTAAGGAAGGTAAACTGAAGTTGGATTTTT
CTGCTTGGAGGCAGGAGTTGACGGAGCAGAAAGTGAAGCACCCGTTG
AACTTTAAAACTTTTGGTGATGCTATTCCTCCGCAATATGCTATCCA
GGTTCTAGATGAGTTAACTAATGGGAATGCTATTATAAGTACTGGTG
TGGGGCAACACCAGATGTGGGCTGCTCAGTACTATAAGTACAGAAAG
CCACGCCAATGGTTGACATCTGGTGGATTAGGAGCAATGGGATTTGG
TTTGCCTGCTGCTATTGGTGCAGCTGTTGGAAGACCGGATGAAGTTG
TGGTTGACATTGATGGCGATGGCAGTTTCATCATGAATGTGCAGGAG
CTTGCAACAATTAAGGTGGAGAATCTCCCAGTTAAGATTATGTTGCT
GAATAATCAACACTTGGGAATGGTGGTTCAActcGAGGATCGGTTCT
ATAAGGCTAACAGAGCACACACATACCTGGGGAATCCTTCTAATGAG
GCGGAGATTTTCCCTAACATGTTGAAATTTGCAGAGGCTTGTGGTGT
ACCTGCTGCAAGAGTGACACATAGGGATGATCTTAGAGCTGCCATTC
AGAAGATGTTAGACACTCCTGGGCCATACTTGTTGGACGTGATTGTA
CCTCATCAGGAACATGTTCTACCTATGATTCCCAGTGGCGGAGCTTT
CAAAGATGTGATCACAGAGGGTGATGGGAGAAGTTCCTATTGAGTTT
GAGAAGCTGCAGAGCTAGTTCTAGACCTTGTATTATCTGATTTTAAA
CTTCTATTAAGCCAAACATGTTCTGTCTATCAGTTTGTTATTAGTTT
TTGCCGTGGCTTTGCTCATTGTCACTGTTGTACTATTAAGTAgggtt
aGTTGATATTTATGATTGCTTTAAGTTTTGCATCATCTCCCTTTGGT
TTTGAATGTGAAGGATTTCAGCAAAGTTCATTCTCTATTTGCAACAT
CCACTTGGTATCTGGAGATTAATTTCTAGTGGAGTAGTTTAGTGCGA
TAAAGTTAGCTTGTTTCCACATTTTTATTTCGTAAGCTATGTCAGCC
AGGGTCAGATTGGAACTAAAGGTGTTAAATGGGTGGGTCGGGCCGGG
CTTCTATTTACTAGTCaaaaattcaaatagaggacctaacagaactc
gccgtaaagactggcgaacagttcatacagagtctcttacgactcaa
tgacaagaagaaaatcttcgtcaacatggtggagcacgacacacttg
tctactccaaaaatatcaaagatacagtctcagaagaccaaagggca
attgagacttttcaacaaagggtaatatccggaaacctcctcggatt
ccattgcccagctatctgtcactttattgtgaagatagtggaaaagg
aaggtggctcctacaaatgccatcattgcgataaaggaaaggccatc
gttgaagatgcctctgccgacagtggtcccaaagatggacccccacc
cacgaggagcatcgtggaaaaagaagacgttccaaccacgtcttcaa
agcaagtggattgatgtgatatctccactgacgtaagggatgacgca
caatcccactatccttcgcaagacccttcctctatataaggaagttc
atttcatttggagagaacaGGATCCAtgaaaaagcctgaactcaccg
cgacgtctgtcgagaagtttctgatcgaaaagttcgacagcgtctcc
gacctgatgcagctctcggagggcgaagaatctcgtgctttcagctt
cgatgtaggagggcgtggatatgtcctgcgggtaaatagctgcgccg
atggtttctacaaagatcgttatgtttatcggcactttgcatcggcc
gcgctcccgattccggaagtgcttgacattggggagtttagcgagag
cctgacctattgcatctcccgccgtgcacagggtgtcacgttgcaag
acctgcctgaaaccgaactgcccgctgttctacaaccggtcgcggag
gctatggatgcgatcgctgcggccgatcttagccagacgagcgggtt
cggcccattcggaccgcaaggaatcggtcaatacactacatggcgtg
atttcatatgcgcgattgctgatccccatgtgtatcactggcaaact
gtgatggacgacaccgtcagtgcgtccgtcgcgcaggctctcgatga
gctgatgctttgggccgaggactgccccgaagtccggcacctcgtgc
acgcggatttcggctccaacaatgtcctgacggacaatggccgcata
acagcggtcattgactggagcgaggcgatgttcggggattcccaata
cgaggtcgccaacattttcttctggaggccgtggttggcttgtatgg
agcagcagacgcgctacttcgagcggaggcatccggagcttgcagga
tcgccacgactccgggcgtatatgctccgcattggtcttgaccaact
ctatcagagcttggttgacggcaatttcgatgatgcagcttgggcgc
agggtcgatgcgacgcaatcgtccgatccggagccgggactgtcggg
cgtacacaaatcgcccgcagaagcgcggccgtctggaccgatggctg
tgtagaagtactcgccgatagtggaaaccgacgccccagcactcgtc
cgagggcaaagaaaGGCGCCGagggcagaggaagtcttctaacatgc
ggtgacgtggaggagaatcccggccctAGATCTAtggctcctaagaa
gaagagaaaggttataacaatggtgagcaagggcgaggagctgttca
ccggggtggtgcccatcctggtcgagctggacggcgacgtaaacggc
cacaagttcagcgtgtccggcgagggcgagggcgatgccacctacgg
caagctgaccctgaagttcatctgcaccaccggcaagctgcccgtgc
cctggcccaccctcgtgaccaccttcggctacggcctgcagtgcttc
gcccgctaccccgaccacatgaagcagcacgacttcttcaagtccgc
catgcccgaaggctacgtccaggagcgcaccatcttcttcaaggacg
acggcaactacaagacccgcgccgaggtgaagttcgagggcgacacc
ctggtgaaccgcatcgagctgaagggcatcgacttcaaggaggacgg
caacatcctggggcacaagctggagtacaactacaacagccacaacg
tctatatcatggccgacaagcagaagaacggcatcaaggtgaacttc
aagatccgccacaacatcgaggacggcagcgtgcagctcgccgacca
ctaccagcagaacacccccatcggcgacggccccgtgctgctgcccg
acaaccactacctgagctaccagtccgccctgagcaaagaccccaac
gagaagcgcgatcacatggtcctgctggagttcgtgaccgccgccgg
gatcactctcggcatggacgagctgtacaagccgcggttcccgggag
aCctttagCTAGCTtcaaacatttggcaataaagtttcttaagattg
aatcctgttgccggtcttgcgatgattatcatataatttctgttgaa
ttacgttaagcatgtaataattaacatgtaatgcatgacgttattta
tgagatgggtttttatgattagagtcccgcaattatacatttaatac
gcgatagaaaacaaaatatagcgcgcaaactaggataaattatcgcg
cgcggtgtcatctatgttactagatcggaaattcgtaatcatggtca
tagcATGCTGGCTTCGGGTTCATCCGGGTAAAAATTGAACCATAAGG
GTTACTGGTTGAGGGGGCCGGATCGTGCCGGGTTTAGTGTATTTTTA
AATTTTTTTTTTAGAATTTTGTATAACTATTGTAAGTTATATTAATA
CAAAGTATTAACATAAAAAACACAAGGAAGATGGGTAAAAAATTGCA
ATTATTGCAAGTGGTACATTTATTTCATAATTTAAAGTTTCAAACTT
ACAAATTGAAAGGTTTACATTTTTAACAAGTAAATTTAAAGGTTTTG
CATTGCCCTTGTAAGTTCGTCATAAGCAATATGAACTGATTGACCTT
CTTCTGGAATATTAAATTCGGATGGGTTACCATGTGTTAATATATCT
CCAAGTTTCTCGTCTTCTAGGCTATCAACATCTTCATGTCCTTGATT
TCTTCGTTTCAATCTAATCCAATCTCTGAAACATACTAAAACTTCCA
AAGCATTGCCTGCCAATGAGTGATGGATGTCTCCAAGTTGTTGTCTT
GCTTGGTTAAATGCACTCTCCGATGCAAGCCTAGAAATTGACACATT
CAGCACGTCCTGAGCCATAGCGAAAAGAATAGTAAATTGCTTTCTAT
TCTCATGCCACCATCCCAACAGTGAAAATTCCTTTGTGCGAGGTTCT
TTTTGCTTCTGCAAGTAAAATTGAAGTTCATCAATGTTCTTGCTACT
AaTTTGAGTGTTAGAAAATGTAGAAAAAATATTAATACTATCAAGCA
TTCATCATCATCCACATGGCATAGTGGGATTAATACTGCCTATAGCA
CGAGCAAACATCATCAATTATATTTGCATAATAATTATATAATTATT
GTAAATAATCATTTAGCTTGTTCGTAGAAACATATAAATCTGGGGTT
TCAGTTGGTCCAATCTCCATATAAGTACATAAAGCATTCATTAATTG
GTGACAATCAAATATCTTAATAGAAGGATTTAAAACAGCACCAATTA
CGTAAATCGGAGAACTTGAAAAAAAATATTTTTTAAATTTTGCTTGC
ATTTTTCAACAACATCCTTATATTTTTCTTTCTTCTTAAATTTAAAA
AGTAGAAAAAAATTTCAGCTATATGTATTAAAACCATAGTAACAATA
TGATAATATGCTCCAAAAAACTCAACAGTAGCTGTATAAAATTTATG
TAAAAATTTAACATCATTAATGGCCTCCCAAGCTT
SEQ ID NO. 6: Donor sequence for ALS2 knock-in
(HPH and YFP; the underlined sequences represent
the homology arms for homologous recombination)
GAATTCGATATTGGAGAGTAAGGAAGGTAAACTGAAGTTGGATTTTT
CTGCTTGGAGGCAGGAGTTGATGGAGCAGAAAGTGAAGCACCCGTTG
AACTTTAAAACTTTTGGTGATGCTATTCCTCCACAATATGCTATCCA
GGTTCTAGATGAGTTAACTAATGGGAATGCTATTATAAGTACTGGTG
TGGGGCAACACCAGATGTGGGCTGCTCAATACTATAAGTACAGAAAG
CCACGCCAATGGTTGACATCTGGTGGATTAGGAGCAATGGGATTTGG
TTTACCCGCTGCTATTGGTGCAGCTGTGGGAAGACCGGATGAAGTTG
TGGTTGACATTGATGGCGATGGCAGTTTCATCATGAATGTGCAGGAG
CTGGCAACAATTAAGGTGGAGAATCTCCCAGTTAAGATTATGTTACT
GAATAATCAACACTTGGGAATGGTGGTTCAACTCGAGGATCGGTTCT
ATAAGGCTAACAGAGCACACACATACCTGGGGAATCCTTCTAATGAG
GCGGAAATCTTTCCTAATATGTTGAAATTTGCAGAGGCTTGTGGTGT
ACCTGCTGCAAGGGTGACACATAGGGATGATCTTAGAGCTGCCATTC
AGAAGATGTTAGACACTCCTGGGCCATACTTGTTGGATGTGATTGTA
CCTCATCAGGAACATGTTCTACCTATGATTCCCAGTGGCGGAGCTTT
CAAAGATGTGATCACAGAGGGTGACGGGAGAATTTCCTATTGAGTTT
GAGAAGCTGCAGAGCTAGTTCTAGGCGTCTAGGCCTTGTATTATCTA
AAATAAACTTCTATTAAGCCAAACATGTTCTGTCTATTAGTTTGTTA
TTAGTTTTTGCCGTGGCTTTGCTCATCGTCACTGTTGTACTATTAAG
TAGTTGATATTTATGTTTGTTTTGCATCATCCCCCTTTGGTTTTGAA
TGTGAAGGATTTCAGCAAAGTTTCATCCTCTATTTGCAACAATCTGG
AGATTAATTTCTAATGGAGTAGTTTAGTGTAATAAAGACTAGTCaaa
gattcaaatagaggacctaacagaactcgccgtaaagactggcgaac
agttcatacagagtctcttacgactcaatgacaagaagaaaatcttc
gtcaacatggtggagcacgacacacttgtctactccaaaaatatcaa
agatacagtctcagaagaccaaagggcaattgagacttttcaacaaa
gggtaatatccggaaacctcctcggattccattgcccagctatctgt
cactttattgtgaagatagtggaaaaggaaggtggctcctacaaatg
ccatcattgcgataaaggaaaggccatcgttgaagatgcctctgccg
acagtggtcccaaagatggacccccacccacgaggagcatcgtggaa
aaagaagacgttccaaccacgtcttcaaagcaagtggattgatgtga
tatctccactgacgtaagggatgacgcacaatcccactatccttcgc
aagacccttcctctatataaggaagttcatttcatttggagagaaca
GGATCCAtgaaaaagcctgaactcaccgcgacgtctgtcgagaagtt
tctgatcgaaaagttcgacagcgtctccgacctgatgcagctctcgg
agggcgaagaatctcgtgctttcagcttcgatgtaggagggcgtgga
tatgtcctgcgggtaaatagctgcgccgatggtttctacaaagatcg
ttatgtttatcggcactttgcatcggccgcgctcccgattccggaag
tgcttgacattggggagtttagcgagagcctgacctattgcatctcc
cgccgtgcacagggtgtcacgttgcaagacctgcctgaaaccgaact
gcccgctgttctacaaccggtcgcggaggctatggatgcgatcgctg
cggccgatcttagccagacgagcgggttcggcccattcggaccgcaa
ggaatcggtctatacactacatggcgtgatttcatatgcgcgattgc
tgatccccatgtgtatcactggcaaactgtgatggacgacaccgtca
gtgcgtccgtcgcgcaggctctcgatgagctgatgctttgggccgag
gactgccccgaagtccggcacctcgtgcacgcggatttcggctccaa
caatgtcctgacggacaatggccgcataacagcggtcattgactgga
gcgaggcgatgttcggggattcccaatacgaggtcgccaacatcttc
ttctggaggccgtggttggcttgtatggagcagcagacgcgctactt
cgagcggaggcatccggagcttgcaggatcgccacgactccgggcgt
atatgctccgcattggtcttgaccaactctatcagagcttggttgac
ggcaatttcgatgatgcagcttgggcgcagggtcgatgcgacgcaat
cgtccgatccggagccgggactgtcgggcgtacacaaatcgcccgca
gaagcgcggccgtctggaccgatggctgtgtagaagtactcgccgat
agtggaaaccgacgccccagcactcgtccgagggcaaagaaaGGCGC
CGagggcagaggaagtcttctaacatgcggtgacgtggaggagaatc
ccggccctAGATCTAtggctcctaagaagaagagaaaggttataaca
atggtgagcaagggcgaggagctgttcaccggggtggtgcccatcct
ggtcgagctggacggcgacgtaaacggccacaagttcagcgtgtccg
gcgagggcgagggcgatgccacctacggcaagctgaccctgaagttc
atctgcaccaccggcaagctgcccgtgccctggcccaccctcgtgac
caccttcggctacggcctgcagtgcttcgcccgctaccccgaccaca
tgaagcagcacgacttcttcaagtccgccatgcccgaaggctacgtc
caggagcgcaccatcttcttcaaggacgacggcaactacaagacccg
cgccgaggtgaagttcgagggcgacaccctggtgaaccgcatcgagc
tgaagggcatcgacttcaaggaggacggcaacatcctggggcacaag
ctggagtacaactacaacagccacaacgtctatatcatggccgacaa
gcagaagaacggcatcaaggtgaacttcaagatccgccacaacatcg
aggacggcagcgtgcagctcgccgaccactaccagcagaacaccccc
atcggcgacggccccgtgctgctgcccgacaaccactacctgagcta
ccagtccgccctgagcaaagaccccaacgagaagcgcgatcacatgg
tcctgctggagttcgtgaccgccgccgggatcactctcggcatggac
gagctgtacaagccgcggttcccgggagaCctttagCTAGCTtcaaa
catttggcaataaagtttcttaagattgaatcctgttgccggtcttg
cgatgattatcatataatttctgttgaattacgttaagcatgtaata
attaacatgtaatgcatgacgttatttatgagatgggtttttatgat
tagagtcccgcaattatacatttaatacgcgatagaaaacaaaatat
agcgcgcaaactaggataaattatcgcgcgcggtgtcatctatgtta
ctagatcggaaattcgtaatcatggtcatagcATGCTTGGAACTTCC
TCTTTAGGTTGGATGTAATCCCTATTAGGGCTTTCTCTTAATTTTAT
TATTGAATTGTTGGCTTTTAATCTGAGCAAGTTGATTTGCAGCTTTC
TCTCGAGTCCTAGGAGCAATACGTTATCTCTGTCTCCTATTTCCTAG
TGGATAATCTTATGATGGAAATATGTGGAGTTAGGAAACTGTTGACT
GCTAAATTTCTCTTTGTGAGGCGTCTGACAGGTATGCTTTCAATCTA
TAGCAGTTTGATCAGACTTTGTTTACGTATAACAATGTTACGCAAAC
AAACACGTGCTTTTTAAACAGTTATAGGTGCTTAGCTACCGACAATA
CATCACATATAACAGGTACATGTATATCTGGCGTTTTGCTTTTAAAT
AGTACATTTCATTTTTGTATTATGCACTGACCAGACCCTGTTTATGG
GGTTTGTTGTTGTGTTATTCACTGAATCTTTAACATTCAATCTTCAT
GAGAAACTATTCTTTACGGCGTCTAATGTTCTTTCTACTAAACAACC
AAGTCTTTGTACCTAACACACATTGTAATTGATCACTAGAAACTTGT
CAAGTTGCTGATTTAGTAATCTATTTTCTTATAATGAAGATGGAACT
TATCATTCCCAAAAATATATCCTCCTTTTGTTTTCAAGGTTACAAAT
TCTCTAGAAAATCATTTCATGTGGAGTAGCTAGTATCTTTAAACATT
AAGTAATTATCTCCTGAGTTCTGCCTGCCTCTTATATTTCTTTGGTG
ATTCCTCTTTTTTTAGGGGTGCCGTGCTAGGGGATATTTTTTGTGGA
GCAATCCTTTTGCGGAACTACTTATATTCAATATATTAAGTATTATT
GGTTTATTTCTTTTAAAATCCATATTTGATTTCACAACCATAATCGG
GTAATTCATGATACCCATGAATATTTCTATCAAATTCTTAATGCTTC
TATATAAGCACAATTGTGATTTTACTCGACTTTGAGCATGTCTTCAA
AGTTGAAAATTTAGGTGTTTCTTGCATGGTGTTATAGCTGTCAAAGT
GGTGTTAGGGATGAAAAGTTTTGCGGATGAGGGAGAGCTCTGCATGG
CGTAGAAGGTCACCAAACATGTCTCCTCTCTCTATTTCTACTAGCAT
CGCCTAGAAGCCTATCAATTTGTTGAGAGGACTTATATTACCGAGGA
AGATACAACCGTTTTTAAAGTTAGGAAAAAACATTATTCATAAGTTA
TTTACTATGGTTCTAGGTGATCTTGGTCCATCATAATCAAGTTTTCA
TCTTCTTAATTTCTCTCATTTTTGCTTTGGGGTGTGTCTTAGTTTTC
ATCACAAAGGGAAGAAGATCCATTAGAGCATCACATGTTCTTTGAAC
CTAAGACAAGACTCTTTATTTAACCCCCGACACATTATCCTTCAATG
AAGTTTTCTCCTAGGGAGAGAAGCTT
SEQ ID NO. 7: ALS1 gene sequence
(SurA- N. Tabacum)- Accession no. X07644
SEQ ID NO. 8: ALS2 gene sequence
(SurB- N. Tabacum)- Accession No. X07645
SEQ ID NO. 9: Solanum tuberosum AHAS gene-
Accession No. HM114275
SEQ ID NO. 10: Capsicum Annum AHAS gene-
Accession No. EU616547
SEQ ID NO. 11: Donor sequence for ALS2 knock-in
(YFP only; the underlined sequences represent
the homology arms for homologous recombination)
GATATTGGAGAGTAAGGAAGGTAAACTGAAGTTGGATTTTTCTGCTT
GGAGGCAGGAGTTGATGGAGCAGAAAGTGAAGCACCCGTTGAACTTT
AAAACTTTTGGTGATGCTATTCCTCCACAATATGCTATCCAGGTTCT
AGATGAGTTAACTAATGGGAATGCTATTATAAGTACTGGTGTGGGGC
AACACCAGATGTGGGCTGCTCAATACTATAAGTACAGAAAGCCACGC
CAATGGTTGACATCTGGTGGATTAGGAGCAATGGGATTTGGTTTACC
CGCTGCTATTGGTGCAGCTGTGGGAAGACCGGATGAAGTTGTGGTTG
ACATTGATGGCGATGGCAGTTTCATCATGAATGTGCAGGAGCTGGCA
ACAATTAAGGTGGAGAATCTCCCAGTTAAGATTATGTTACTGAATAA
TCAACACTTGGGAATGGTGGTTCAACTCGAGGATCGGTTCTATAAGG
CTAACAGAGCACACACATACCTGGGGAATCCTTCTAATGAGGCGGAA
ATCTTTCCTAATATGTTGAAATTTGCAGAGGCTTGTGGTGTACCTGC
TGCAAGGGTGACACATAGGGATGATCTTAGAGCTGCCATTCAGAAGA
TGTTAGACACTCCTGGGCCATACTTGTTGGATGTGATTGTACCTCAT
CAGGAACATGTTCTACCTATGATTCCCAGTGGCGGAGCTTTCAAAGA
TGTGATCACAGAGGGTGACGGGAGAATTTCCTATTGAGTTTGAGAAG
CTGCAGAGCTAGTTCTAGGCGTCTAGGCCTTGTATTATCTAAAATAA
ACTTCTATTAAGCCAAACATGTTCTGTCTATTAGTTTGTTATTAGTT
TTTGCCGTGGCTTTGCTCATCGTCACTGTTGTACTATTAAGTAGTTG
ATATTTATGTTTGTTTTGCATCATCCCCCTTTGGTTTTGAATGTGAA
GGATTTCAGCAAAGTTTCATCCTCTATTTGCAACAATCTGGAGATTA
ATTTCTAATGGAGTAGTTTAGTGTAATAAAGACTAGTCaaagattca
aatagaggacctaacagaactcgccgtaaagactggcgaacagttca
tacagagtctcttacgactcaatgacaagaagaaaatcttcgtcaac
atggtggagcacgacacacttgtctactccaaaaatatcaaagatac
agtctcagaagaccaaagggcaattgagacttttcaacaaagggtaa
tatccggaaacctcctcggattccattgcccagctatctgtcacttt
attgtgaagatagtggaaaaggaaggtggctcctacaaatgccatca
ttgcgataaaggaaaggccatcgttgaagatgcctctgccgacagtg
gtcccaaagatggacccccacccacgaggagcatcgtggaaaaagaa
gacgttccaaccacgtcttcaaagcaagtggattgatgtgatatctc
cactgacgtaagggatgacgcacaatcccactatccttcgcaagacc
cttcctctatataaggaagttcatttcatttggagagaacaGGATCT
Atggctcctaagaagaagagaaaggttataacaatggtgagcaaggg
cgaggagctgttcaccggggtggtgcccatcctggtcgagctggacg
gcgacgtaaacggccacaagttcagcgtgtccggcgagggcgagggc
gatgccacctacggcaagctgaccctgaagttcatctgcaccaccgg
caagctgcccgtgccctggcccaccctcgtgaccaccttcggctacg
gcctgcagtgcttcgcccgctaccccgaccacatgaagcagcacgac
ttcttcaagtccgccatgcccgaaggctacgtccaggagcgcaccat
cttcttcaaggacgacggcaactacaagacccgcgccgaggtgaagt
tcgagggcgacaccctggtgaaccgcatcgagctgaagggcatcgac
ttcaaggaggacggcaacatcctggggcacaagctggagtacaacta
caacagccacaacgtctatatcatggccgacaagcagaagaacggca
tcaaggtgaacttcaagatccgccacaacatcgaggacggcagcgtg
cagctcgccgaccactaccagcagaacacccccatcggcgacggccc
cgtgctgctgcccgacaaccactacctgagctaccagtccgccctga
gcaaagaccccaacgagaagcgcgatcacatggtcctgctggagttc
gtgaccgccgccgggatcactctcggcatggacgagctgtacaagcc
gcggttcccgggagaCctttagCTAGCTtcaaacatttggcaataaa
gtttcttaagattgaatcctgttgccggtcttgcgatgattatcata
taatttctgttgaattacgttaagcatgtaataattaacatgtaatg
catgacgttatttatgagatgggtttttatgattagagtcccgcaat
tatacatttaatacgcgatagaaaacaaaatatagcgcgcaaactag
gataaattatcgcgcgcggtgtcatctatgttactagatcggaaatt
cgtaatcatggtcatagcATGCTTGGAACTTCCTCTTTAGGTTGGAT
GTAATCCCTATTAGGGCTTTCTCTTAATTTTATTATTGAATTGTTGG
CTTTAATCTGAGCAAGTTGATTTGCAGCTTTCTCTCGAGTCCTAGGA
GCAATACGTTATCTCTGTCTCCTATTTCCTAGTGGATAATCTTATGA
TGGAAATATGTGGAGTTAGGAAACTGTTGACTGCTAAATTTCTCTTT
GTGAGGCGTCTGACAGGTATGCTTTCAATCTATAGCAGTTTGATCAG
ACTTTGTTTACGTATAACAATGTTACGCAAACAAACACGTGCTTTTT
AAACAGTTATAGGTGCTTAGCTACCGACAATACATCACATATAACAG
GTACATGTATATCTGGCGTTTTGCTTTTAAATAGTACATTTCATTTT
TGTATTATGCACTGACCAGACCCTGTTTATGGGGTTTGTTGTTGTGT
TATTCACTGAATCTTTAACATTCAATCTTCATGAGAAACTATTCTTT
ACGGCGTCTAATGTTCTTTCTACTAAACAACCAAGTCTTTGTACCTA
ACACACATTGTAATTGATCACTAGAAACTTGTCAAGTTGCTGATTTA
GTAATCTATTTTCTTATAATGAAGATGGAACTTATCATTCCCAAAAA
TATATCCTCCTTTTGTTTTCAAGGTTACAAATTCTCTAGAAAATCAT
TTCATGTGGAGTAGCTAGTATCTTTAAACATTAAGTAATTATCTCCT
GAGTTCTGCCTGCCTCTTATATTTCTTTGGTGATTCCTCTTTTTTTA
GGGGTGCCGTGCTAGGGGATATTTTTTGTGGAGCAATCCTTTTGCGG
AACTACTTATATTCAATATATTAAGTATTATTGGTTTATTTCTTTTA
AAATCCATATTTGATTTCACAACCATAATCGGGTAATTCATGATACC
CATGAATATTTCTATCAAATTCTTAATGCTTCTATATAAGCACAATT
GTGATTTTACTCGACTTTGAGCATGTCTTCAAAGTTGAAAATTTAGG
TGTTTCTTGCATGGTGTTATAGCTGTCAAAGTGGTGTTAGGGATGAA
AAGTTTTGCGGATGAGGGAGAGCTCTGCATGGCGTAGAAGGTCACCA
AACATGTCTCCTCTCTCTATTTCTACTAGCATCGCCTAGAAGCCTAT
CAATTTGTTGAGAGGACTTATATTACCGAGGAAGATACAACCGTTTT
TAAAGTTAGGAAAAAACATTATTCATAAGTTATTTACTATGGTTCTA
GGTGATCTTGGTCCATCATAATCAAGTTTCATCTTCTTAATTTCTCT
CATTTTTGCTTTGGGGTGTGTCTTAGTTTTCATCACAAAGGGAAGAA
GATCCATTAGAGCATCACATGTTCTTTGAACCTAAGACAAGACTCTT
TATTTAACCCCCGACACATTATCCTTCAATGAAGTTTTCTCCTAGGG
AGAG
SEQ ID NO. 12: Forward primer recognizing the
tobacco ALS gene for target insertion genotyping
SEQ ID NO. 13: Reverse primer recognizing the
NOS terminator for target insertion genotyping

### REFERENCES

Belhaj K., Chaparro-Garcia A., Kamoun S., Nekrasov V. (2013) Plant genome editing made easy: targeted mutagenesis in model and crop plants using the CRISPR/Cas system. Plant Methods. 9:39-49.
Cermak T, Doyle E L, Christian M, Wang L, Zhang Y, et al. (2011) Efficient design and assembly of custom TALEN and other TAL effector-based constructs for DNA targeting. Nucleic Acids Res 39: e82.
Christian M, Cermak T, Doyle E L, Schmidt C, Zhang F, Hummel A, Bogdanove A J, Voytas D F (2010) Targeting DNA double-strand breaks with TAL effector nucleases. Genetics 186:757-761.
Christou, P. (1997) Rice transformation: bombardment. Plant Mol Biol. 35(1-2):197-203.
Doyle E L, Booher N J, Standage D S, Voytas D F, Brendel V P, et al. (2012) TAL Effector-Nucleotide Targeter (TALE-NT) 2.0: tools for TAL effector design and target prediction. Nucleic Acids Res 40: W117-122.
Hirel B, Marsolier M C, Hoarau A, Hoarau J, Brangeon J, Schafer R, Verma D P. (1992) Forcing expression of a soybean root glutamine synthetase gene in tobacco leaves induces a native gene encoding cytosolic enzyme. Plant Mol Biol. 20(2):207-18.
Jinek, M., Chylinski, K., Fonfara I., Hauer M., Doudna, J A., Charpentier E. (2012) A Programmable Dual-RNA-Guided DNA endonuclease in Adaptative Bacterial Immunity 337:816-821.
Kawamata S, Shimoharai K, Imura Y, Ozaki M, Ichinose Y, Shiraishi T, Kunoh H, Yamada T. (1997) Temporal and spatial pattern of expression of the pea phenylalanine ammonia-lyase gene1 promoter in transgenic tobacco. Plant Cell Physiol. 38(7):792-803.
Klein T M, Arentzen R, Lewis P A, Fitzpatrick-McElligott S. (1992) Transformation of microbes, plants and animals by particle bombardment. Biotechnology (N Y). 10(3):286-91.
Laursen C M, Krzyzek R A, Flick C E, Anderson P C, Spencer T M. (1994) Production of fertile transgenic maize by electroporation of suspension culture cells. Plant Mol Biol. 24(1):51-61.
Lloyd, D. Plaisier C L, Carroll D, Drews G N, (2005) Targeted mutagenesis using zinc-finger nucleases in Arabidopsis. Proc. Natl. Acad. Sci. USA 10:2232-2237
Mussolino C, Morbitzer R, Lutge F, Dannemann N, Lahaye T, et al. (2011) A novel TALE nuclease scaffold enables high genome editing activity in combination with low toxicity. Nucleic Acids Res 39: 9283-9293.
Reyon D, Tsai S Q, Khayter C, Foden J A, Sander J D, et al. (2012) FLASH assembly of TALENs for high-throughput genome editing. Nat Biotechnol 30: 460-465.
Schena M, Lloyd A M, Davis R W. (1991) A steroid-inducible gene expression system for plant cells. Proc Natl Acad Sci USA. 88(23):10421-5.
Thompson G A, Larkins B A. (1989) Structural elements regulating zein gene expression. Bioessays. 10(4):108-13.
Townsend J A, Wright D A, Winfrey R J, Fu F, Maeder M L, Joung J K, Voytas D F (2009) High-frequency modification of plant genes using engineered zinc-finger nucleases. Nature. 459: 442-445
Van den Elzen P J M, Townsend J, Lee K Y, Bedbrook J R (1985) A chimaeric hygromycin resistance gene as a selectable marker in plant cells. Plant Molecular Biology. 5: 299-302.
Wright D A, Townsend J A, Winfrey R J, Jr., Irwin P A, Rajagopal J, et al. (2005) High-frequency homologous recombination in plants mediated by zinc-finger nucleases. Plant J. 44:693-705.
Yamamoto Y Y, Kondo Y, Kato A, Tsuji H, Obokata J. (1997) Light-responsive elements of the tobacco PSI-D gene are located both upstream and within the transcribed region. Plant J. 12(2):255-65.
Yoo S D, Cho Y H, Sheen J (2007) Arabidopsis mesophyll protoplasts: a versatile cell system for transient gene expression analysis. Nature protocols. 2:1565-1572.
Zhang F, Cong L, Lodato S, Kosuri S, Church G M, et al. (2011) Efficient construction of sequence-specific TAL effectors for modulating mammalian transcription. Nat Biotechnol. 29:149-153.

1. A method for targeted genetic insertion into a plant genome without inserting an exogenous selectable marker into said genome comprising:
a) providing a plant cell which comprises an endogenous gene that can be modified to confer herbicide resistance;
b) obtaining a donor matrix comprising a sequence homologous to said endogenous gene, said homologous sequence including a genetic modification to render said gene capable of conferring herbicide resistance to the cell, and downstream of said homologous sequence, a desired transgene to be inserted into the genome;
c) transformation of the plant with said donor matrix
d) further transforming said plant cell with a nucleic acid expressing a sequence-specific nuclease to specifically cleave said gene susceptible to confer herbicide resistance;
e) expressing said sequence-specific nuclease into said cell in order to induce homologous recombination between the endogenous gene and the donor matrix;
to produce a plant cell having resistance to herbicide, in which stable integration of the transgene has occurred downstream of the endogenous gene conferring said resistance.

2. The method of claim 1, wherein the sequence-specific nuclease is a meganuclease.

3. The method of claim 2, wherein the meganuclease is a TALEN (TAL Effector nuclease).

4. The method of claim 2, wherein the meganuclease is a homing endonuclease.

5. The method of claim 2, wherein the meganuclease is a ZFN (Zinc Finger Nuclease).

6. The method of claim 1, wherein the endogenous plant gene expresses ALS (acetolactate synthase).

7. The method of claim 1, wherein the endogenous plant gene has at least 75%, preferably at least 80%, more preferably at least 90%, even more preferably at least 95% identity with SEQ ID NO. 7 or SEQ ID NO. 8.

8. The method of claim 6, wherein said sequence homologous to said endogenous gene comprised on said matrix allows the expression of a functional ALS protein by the cell after homologous recombination.

9. The method of claim 6, wherein said ALS protein is functional and has a mutation corresponding to P191A, W568L, or S647T.

10. The method of claim 1, wherein the cell in which the transgene is inserted is selected on the resistance to herbicide conferred by the modified endogenous gene.

11. The method of claim 10, wherein said herbicide is sulfonylurea, such as chlorsulfuron, or an imidazolinone herbicide.

12. The method of claim 1, wherein at least two endogenous genes are selected for transgene insertions.

13. The method of claim 7, wherein at least two genes having identity with ALS genes are used for transgene insertions.

14. The method of claim 13, wherein said two genes are respectively ALS1 and ALS2.

15. The method of claim 1, wherein expression of the transgene is regulated by a constitutive promoter, such as the Cauliflower Mosaic Virus 35S promoter.

16. The method of claim 1, wherein the expression of the transgene is regulated by an inducible promoter, such as the steroid-inducible glucocorticoid responsive promoter.

17. The method of claim 1, wherein the expression of the transgene is regulated by a tissue specific promoter.

18. The method of claim 1, wherein the transgene encodes for a therapeutic protein, such as a vaccine.

19. The method of claim 1, wherein said donor matrix comprises a pair of left and right arms, said arms having homology to the genetic locus to be targeted.

20. The method of claim 19, wherein at least one arm contains at least one engineered mutation to permit mutation of the endogenous plant gene by homologous recombination.

21. The method of claim 1, wherein said donor matrix comprises one or more additional nuclease cleavage sites for the insertion of one or more additional transgenes subsequent to the initial plant transformation.

22. The method of claim 1, wherein said donor matrix is encoded by a plasmid vector.

23. The method of claim 1, wherein said donor matrix is encoded by an episomal vector.

24. The method of claim 1, wherein said plant species is a field crop, such as but not limited to alfalfa, barley, bean, corn, cotton, flax, pea, rape, rice, rye, safflower, sorghum, soybean, sunflower, tobacco, wheat.

25. The method of claim 1, wherein said plant genus is Nicotiana.

26. The method of claim 1, wherein said plant species is a vegetable crop, such as but not limited to asparagus, beet, broccoli, cabbage, carrot, cauliflower, celery, cucumber, eggplant, lettuce, onion, pepper, potato, pumpkin, radish, spinach, squash, taro, tomato, and zucchini.

27. The method of claim 1, wherein said plant species is a fruit crop, such as but not limited to almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon.

28. The method of claim 1, wherein said plant species is an ornamental, such as but not limited to alder, ash, aspen, azalea, birch, boxwood, camellia, carnation, chrysanthemum, elm, fir, ivy, jasmine, juniper, oak, palm, poplar, pine, redwood, rhododendron, rose, and rubber.

29. The method of claim 1, wherein transformation is effected through insertion of the donor matrix construct into isolated plant protoplasts.

30. The method of claim 1, wherein transformation is effected through insertion of the donor matrix construct into isolated plant protoplasts through PEG (polyethylene glycol) mediated transfection.

31. The method of claim 1, wherein transformation is effected through insertion of the donor matrix construct into an isolated plant protoplast through electroporation.

32. The method of claim 1, wherein transformation is effected through insertion of the donor matrix construct into an isolated plant protoplast through biolistic mediated transfection.

33. The method of claim 1, wherein transformation is effected through insertion of the donor matrix construct into an isolated plant protoplast through sonication mediated transfection.

34. The method of claim 1, wherein transformation is effected through insertion of the donor matrix construct into an isolated plant protoplast through liposome mediated transfection.

35. The method of claim 1, wherein transformation is effected through insertion of the donor matrix construct into an isolated plant protoplast through direct DNA uptake transfection, such as but not limited to CaCl₂ uptake transfection.

36. A transformed plant cell obtainable according to the method of claim 1.

37. A herbicide resistant plant grown or cultured from the plant cell of claim 36, a seed thereof, or progeny thereof having herbicide resistance.

38. A transformed plant cell having a transgene in its genome, preferably two transgenes, respectively inserted adjacent to at least one gene having at least 75%, preferably at least 80%, more preferably at least 90%, even more preferably at least 95% identity with an ALS gene, more particularly with SEQ ID NO. 7 or 8.

39. A transformed plant cell according to claim 38, wherein at least one of its ALS proteins displays a mutation corresponding to P191A, W568L, or S647T.

40. A transformed plant cell according to claim 37, wherein said plant is resistant to sulfonylurea or an imidazolinone herbicide.

41. A transformed plant cell according to claim 40, wherein said plant cell is resistant to chlorsulfuron.

42. A transformed plant cell according to claim 38, wherein said plant cell does not comprise any further transgenes in its genome.

43. A transformed plant cell according to claim 38, wherein said transgene does not comprise any exogenous selection marker.

44. A kit for the targeted genetic modification of a plant species comprising a donor matrix as defined into any one of claims 1 to 35 and a vector encoding a meganuclease designed to target an endogenous gene involved into herbicide resistance, and optionally, plant cells having an endogenous gene that can be modified to confer herbicide resistance, reagents, supplies, or equipment for transforming a plant cell, separate containers for each ingredient, packaging materials, and/or instructions for use in preparing a herbicide-resistant plant cell.

45. A vector containing a donor matrix comprising a sequence homologous to an endogenous plant cell gene, said homologous sequence including a genetic modification to render the endogenous plant cell gene capable of conferring herbicide resistance to the cell, and downstream of said homologous sequence, a desired transgene to be inserted into the genome, and optionally, a gene encoding a sequence specific nuclease to specifically cleave said endogenous plant cell gene.

46. A host cell comprising a vector containing a donor matrix comprising a sequence homologous to an endogenous plant cell gene, said homologous sequence including a genetic modification to render said gene capable of conferring herbicide resistance to the cell, and downstream of said homologous sequence, a desired transgene to be inserted into the genome and optionally a gene encoding a sequence specific nuclease to specifically cleave said endogenous plant cell gene.


SEQUENCE LISTING (13 sequences)

SEQ ID NO: 1 - DNA, 259 bp, artificial sequence
  description of artificial sequence synthetic oligonucleotide
gattaatttctagtggagtagtttagtgcgataaagttagcttgtttccacatttttatttcgtaagctatgtcagccagggtcagattggaactaaaggtgttaaatgggtgggtcgggccgggcttctattttttggacttgtatgggttacgatccgggcctgttataagttgattcttaatggcttcgggttcatccgggtaaaaattgaaccataagggttactggttgagggggccggatcgtgccgggttta

SEQ ID NO: 2 - DNA, 259 bp, artificial sequence; identical to SEQ ID NO: 1
  description of artificial sequence synthetic oligonucleotide

SEQ ID NO: 3 - DNA, 264 bp, artificial sequence
  description of artificial sequence synthetic oligonucleotide
gattaatttctaatggagtagtttagtgtaataaagttagcttgttccacatttttatttcataagctatgtcatgctgggtcagattggaacttcctctttaggttggatgtaatccctattagggctttctcttaattttattattgaattgttggcttttaatctgagcaagttgatttgcagctttctctcgagtcctaggagcaatacgttatctctgtctcctatttcctagtggataatcttatgatggaaatatgt

SEQ ID NO: 4 - DNA, 264 bp, artificial sequence; identical to SEQ ID NO: 3
  description of artificial sequence synthetic oligonucleotide

SEQ ID NO: 5 - DNA, 5017 bp, artificial sequence
  description of artificial sequence synthetic DNA
gaattcactattggaaagtaaggaaggtaaactgaagttggatttttctgcttggaggcaggagttgacggagcagaaagtgaagcacccgttgaactttaaaacttttggtgatgctattcctccgcaatatgctatccaggttctagatgagttaactaatgggaatgctattataagtactggtgtggggcaacaccagatgtgggctgctcagtactataagtacagaaagccacgccaatggttgacatctggtggattaggagcaatgggatttggtttgcctgctgctattggtgcagctgttggaagaccggatgaagttgtggttgacattgatggcgatggcagtttcatcatgaatgtgcaggagcttgcaacaattaaggtggagaatctcccagttaagattatgttgctgaataatcaacacttgggaatggtggttcaactcgaggatcggttctataaggctaacagagcacacacatacctggggaatccttctaatgaggcggagattttccctaacatgttgaaatttgcagaggcttgtggtgtacctgctgcaagagtgacacatagggatgatcttagagctgccattcagaagatgttagacactcctgggccatacttgttggacgtgattgtacctcatcaggaacatgttctacctatgattcccagtggcggagctttcaaagatgtgatcacagagggtgatgggagaagttcctattgagtttgagaagctgcagagctagttctagaccttgtattatctgaaataaacttctattaagccaaacatgttctgtctatcagtttgttattagtttttgccgtggctttgctcattgtcactgttgtactattaagtagggttagttgatatttatgattgctttaagttttgcatcatctccctttggttttgaatgtgaaggatttcagcaaagtttcattctctatttgcaacatccacttggtatctggagattaatttctagtggagtagtttagtgcgataaagttagcttgtttccacatttttatttcgtaagctatgtcagccagggtcagattggaactaaaggtgttaaatgggtgggtcgggccgggcttctatttactagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacaggatccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaaggcgccgagggcagaggaagtcttctaacatgcggtgacgtggaggagaatcccggccctagatctatggctcctaagaagaagagaaaggttataacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagccgcggttcccgggagacctttagctagcttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaaattcgtaatcatggtcatagcatgctggcttcgggttcatccgggtaaaaattgaaccataagggttactggttgagggggccggatcgtgccgggtttagtgtatttttaaattttttttttagaattttgtataactattgtaagttatattaatacaaagtattaacataaaaaacacaaggaagatgggtaaaaaattgcaattattgcaagtggtacatttatttcataatttaaagtttcaaacttacaaattgaaaggtttacatttttaacaagtaaatttaaaggttttgcattgcccttgtaagttcgtcataagcaatatgaactgattgaccttcttctggaatattaaattcggatgggttaccatgtgttaatatatctccaagtttctcgtcttctaggctatcaacatcttcatgtccttgatttcttcgtttcaatctaatccaatctctgaaacatactaaaacttccaaagcattgcctgccaatgagtgatggatgtctccaagttgttgtcttgcttggttaaatgcactctccgatgcaagcctagaaattgacacattcagcacgtcctgagccatagcgaaaagaatagtaaattgctttctattctcatgccaccatcccaacagtgaaaattcctttgtgcgaggttctttttgcttctgcaagtaaaattgaagttcatcaatgttcttgctactaatttgagtgttagaaaatgtagaaaaaatattaatactatcaagcattcatcatcatccacatggcatagtgggattaatactgcctatagcacgagcaaacatcatcaattatatttgcataataattatataattattgtaaataatcatttagcttgttcgtagaaacatataaatctggggtttcagttggtccaatctccatataagtacataaagcattcattaattggtgacaatcaaatatcttaatagaaggatttaaaacagcaccaattacgtaaatcggagaacttgaaaaaaaatattttttaaattttgcttgcatttttcaacaacatccttatatttttctttcttcttaaatttaaaaagtagaaaaaaatttcagctatatgtattaaaaccatagtaacaatatgataatatgctccaaaaaactcaacagtagctgtataaaatttatgtaaaaatttaacatcattaatggcctcccaagctt

SEQ ID NO: 6 - DNA, 5149 bp, artificial sequence
  description of artificial sequence synthetic DNA
gaattcgatattggagagtaaggaaggtaaactgaagttggatttttctgcttggaggcaggagttgatggagcagaaagtgaagcacccgttgaactttaaaacttttggtgatgctattcctccacaatatgctatccaggttctagatgagttaactaatgggaatgctattataagtactggtgtggggcaacaccagatgtgggctgctcaatactataagtacagaaagccacgccaatggttgacatctggtggattaggagcaatgggatttggtttacccgctgctattggtgcagctgtgggaagaccggatgaagttgtggttgacattgatggcgatggcagtttcatcatgaatgtgcaggagctggcaacaattaaggtggagaatctcccagttaagattatgttactgaataatcaacacttgggaatggtggttcaactcgaggatcggttctataaggctaacagagcacacacatacctggggaatccttctaatgaggcggaaatctttcctaatatgttgaaatttgcagaggcttgtggtgtacctgctgcaagggtgacacatagggatgatcttagagctgccattcagaagatgttagacactcctgggccatacttgttggatgtgattgtacctcatcaggaacatgttctacctatgattcccagtggcggagctttcaaagatgtgatcacagagggtgacgggagaatttcctattgagtttgagaagctgcagagctagttctaggcgtctaggccttgtattatctaaaataaacttctattaagccaaacatgttctgtctattagtttgttattagtttttgccgtggctttgctcatcgtcactgttgtactattaagtagttgatatttatgtttgttttgcatcatccccctttggttttgaatgtgaaggatttcagcaaagtttcatcctctatttgcaacaatctggagattaatttctaatggagtagtttagtgtaataaagactagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacaggatccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaaggcgccgagggcagaggaagtcttctaacatgcggtgacgtggaggagaatcccggccctagatctatggctcctaagaagaagagaaaggttataacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagccgcggttcccgggagacctttagctagcttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaaattcgtaatcatggtcatagcatgcttggaacttcctctttaggttggatgtaatccctattagggctttctcttaattttattattgaattgttggcttttaatctgagcaagttgatttgcagctttctctcgagtcctaggagcaatacgttatctctgtctcctatttcctagtggataatcttatgatggaaatatgtggagttaggaaactgttgactgctaaatttctctttgtgaggcgtctgacaggtatgctttcaatctatagcagtttgatcagactttgtttacgtataacaatgttacgcaaacaaacacgtgctttttaaacagttataggtgcttagctaccgacaatacatcacatataacaggtacatgtatatctggcgttttgcttttaaatagtacatttcatttttgtattatgcactgaccagaccctgtttatggggtttgttgttgtgttattcactgaatctttaacattcaatcttcatgagaaactattctttacggcgtctaatgttctttctactaaacaaccaagtctttgtacctaacacacattgtaattgatcactagaaacttgtcaagttgctgatttagtaatctattttcttataatgaagatggaacttatcattcccaaaaatatatcctccttttgttttcaaggttacaaattctctagaaaatcatttcatgtggagtagctagtatctttaaacattaagtaattatctcctgagttctgcctgcctcttatatttctttggtgattcctctttttttaggggtgccgtgctaggggatattttttgtggagcaatccttttgcggaactacttatattcaatatattaagtattattggtttatttcttttaaaatccatatttgatttcacaaccataatcgggtaattcatgatacccatgaatatttctatcaaattcttaatgcttctatataagcacaattgtgattttactcgactttgagcatgtcttcaaagttgaaaatttaggtgtttcttgcatggtgttatagctgtcaaagtggtgttagggatgaaaagttttgcggatgagggagagctctgcatggcgtagaaggtcaccaaacatgtctcctctctctatttctactagcatcgcctagaagcctatcaatttgttgagaggacttatattaccgaggaagatacaaccgtttttaaagttaggaaaaaacattattcataagttatttactatggttctaggtgatcttggtccatcataatcaagttttcatcttcttaatttctctcatttttgctttggggtgtgtcttagttttcatcacaaagggaagaagatccattagagcatcacatgttctttgaacctaagacaagactctttatttaacccccgacacattatccttcaatgaagttttctcctagggagagaagctt

SEQ ID NO: 7 - DNA, 2468 bp, Nicotiana Tabacum
  ALS1 gene sequence (SurA)
ccactgggcaaattagcgtgtattagatacactttggaaaggttgagtgtgtaatgtgatttttgttcgcaaaaagtgtgtaatagggatttagtccatagtttagggggtaacttatgtatttataggttaaatgatggcgactaaatagagcgcccgtgcaatttttactattaaagtagtattaaattttcatacgaccctatatctatggctggcaccaaatttcttcacatttggatccctctttcatttgttctcatccatttttgcgattcatgtgcatttaatcagtaggacccctttttagcttagtagtgctctcatgttctcaacttaatattaaaccaaccacactccatctgcattaccctccttccagtttcgtctctccctgccctccccttcaacaatggcggcggcggctccatctccctcttcttccgctttctccaaaaccctatcgccttcctcctccacatcctccaccctcctccctagatcaaccttccctttcccccaccacccccacaagaccaccccaccacccctccacctcacccacactcacattcacattcacagccaacgccgtcgtttcaccatatccaatgtcatttccactaaccaaaaagtttcccagaccgaaaaaaccgaaactttcgtttcccgttttgctcctgacgaacccagaaagggttccgacgttctcgtggaggctctcgaaagagaaggggttacggacgtctttgcgtacccaggtggcgcttccatggagattcaccaagctttgacccgttcaagcatcatccgcaacgtgctgccacgtcacgagcagggcggtgtcttcgccgctgagggttacgcacgcgccaccggatttcccggcgtttgcattgccacctctggccccggcgccaccaatctcgtcagcggcctcgctgacgcgctactggatagcgtccccattgttgctataacaggtcaagtgccacgtaggatgataggtactgatgcttttcaggaaactcctattgttgaggtaactagatcgattaccaagcataattatctcgttatggacgtagaggatattcctagggttgtacgtgaagcttttttcctcgcgagatcgggccggcctggccctattttgattgatgtacctaaggatattcagcaacaattggtgatacctgactgggatcagccaatgaggttacctggttacatgtctaggttacctaaattgcccaatgagatgcttttagaacaaattgttaggcttatttctgagtcaaagaagcctgttttgtatgtggggggtgggtgttcgcaatcgagtgaggacttgagacgattcgtggagctcacgggtatccccgtggcaagtactttgatgggtcttggagcttttccaactggggatgagctttccctttcaatgttgggtatgcatggtactgtttatgctaattatgctgtggacagtagtgatttgttgctcgcatttggggtgaggtttgatgatagagttactggaaagttagaagcttttgctagccgagcaaaaattgttcacattgatattgattcagctgagattggaaagaacaagcagcctcatgtttccatttgtgcagatatcaagttggcgttacagggtttgaattcgatactggagagtaaggaaggtaaactgaagttggatttttctgcttggaggcaggagttgacggagcagaaagtgaagcacccattgaactttaaaacttttggtgatgcaattcctccgcaatatgctatccaggttctagatgagttaactaatgggaatgctattataagtactggtgtggggcaacaccagatgtgggctgctcaatactataagtacagaaagccacgccaatggttgacatctggtggattaggagcaatgggatttggtttgcccgctgctattggtgcggctgttggaagaccggatgaagttgtggttgacattgatggtgatggcagtttcatcatgaatgtgcaggagcttgcaacaattaaggtggagaatctcccagttaagattatgttactgaataatcaacacttgggaatggtggttcaatgggaggatcggttctataaggctaacagagcacacacatacctggggaatccttctaatgaggcggagatctttcctaatatgctgaaatttgcagaggcttgtggcgtacctgctgcaagagtgacacatagggatgatcttagagctgccattcagaagatgttagacactcctgggccatacttgttggatgtgattgtacctcatcaggaacatgttttacctatgattcccagtggcggagctttcaaagatgtgatcacagagggtgacgggagaagttcctattgagtttgagaagctacagagctagattctaggccttgtattatctaaaataaac

SEQ ID NO: 8 - DNA, 2461 bp, Nicotiana Tabacum
  ALS2 gene sequence (SurB)
tatttcttagcttgttttttttttgttctatattgttactttgagctatatttcataacagcattcacattctttttccatagtcttttttcccttttatattttaatttactgaagtaacaaatacttccacttctttcttcttcccaccctcctaaatatatccaacatctcatttttcttttccccaattctcagacattttaatctttcttttctatttattttcttcatattttgatctctcttccatttgttctcatccattttcgctattcacgtgaattcaatcaagtaggaccctttcagtttcgtggcgctctcgtcttctcagcttaatataaaaccaaccacacaccatctacattgccctttcctttcagtttcgtctctcactgctctcattcaacaataatggcggcggctgcggcggctccatctccctctttctccaaaaccctatcgtcctcctcctccaaatcctccaccctcctccctagatccaccttccctttcccccaccacccccacaaaaccaccccaccacccctccacctcacccccacccacattcacagccaacgccgtcgtttcaccatctccaatgtcatttccactacccaaaaagtttccgagacccaaaaagccgaaactttcgtttcccgttttgcccctgacgaacccagaaagggttccgacgttctcgtggaggccctcgaaagagaaggggttacggacgtttttgcgtacccaggcggcgcttccatggagattcaccaagctttgacgcgctcaagcatcatccgcaacgtgctaccacgtcacgagcagggtggtgtcttcgccgctgagggttacgcacgcgccaccggcttccccggcgtttgcattgccacctccggccctggcgccaccaatctcgtcagtggcctcgcggacgccctactggatagcgtccccattgttgctataaccggtcaagtgccacgtaggatgatcggtactgatgcttttcaggaaactccgattgttgaggtaactagatcgattaccaagcataattatctcgttatggacgtagaggatattcctagggttgtacgtgaggcttttttccttgcgagatcgggccggcctggccctgttttgattgatgtacctaaggatattcagcaacaattggtgatacctgactgggatcagccaatgaggttgcctggttacatgtctaggttacctaaattgcccaatgagatgcttttagaacaaattgttaggcttatttctgagtcaaagaagcctgttttgtatgtggggggtgggtgttcgcaatcgagtgaggagttgagacgattcgtggagctcaccggtatccccgtggcaagtactttgatgggtcttggagcttttccaactggggatgagctttccctttcaatgttgggtatgcatggtactgtttatgctaattatgctgtggacagtagtgatttattgctcgcatttggggtgaggtttgatgatagagttactggaaagttagaagcttttgctagccgagcgaaaattgttcacattgatattgattcagctgagattggaaagaacaagcagcctcatgtttccatttgtgcggatatcaagttggcgttacagggtttgaattcgatattggagagtaaggaaggtaaactgaagttggatttttctgcttggaggcaggagttgacggtgcagaaagtgaagtacccgttgaattttaaaacttttggtgatgctattcctccgcaatatgctatccaggttctagatgagttaactaatgggagtgctattataagtaccggtgttgggcagcaccagatgtgggctgctcaatattataagtacagaaagccacgccaatggttgacatctggtggattaggagcgatgggatttggtttgcccgctgctattggtgcggctgttggaagacctgatgaagttgtggttgacattgatggtgatggcagtttcatcatgaatgtgcaggagctagcaactattaaggtggagaatctcccagttaagattatgttactgaataatcaacacttgggaatggtggttcaatgggaggatcggttctataaggctaacagagcacacacatacctggggaatccttctaatgaggcggagatctttcctaatatgttgaaatttgcagaggcttgtggcgtacctgctgcgagagtgacacacagggatgatcttagagcggctattcaaaagatgttagacactcctgggccatacttgttggatgtgattgtacctcatcaggaacatgttctacctatgattcccagtggcggggctttcaaagatgtgatcacagagggtgacgggagaagttcctattgactttgaggtgctacagagctagattctaggccttgtattatctaaaataaac

SEQ ID NO: 9 - DNA, 2122 bp, Solanum tuberosum
  Solanum tuberosum AHAS gene
tagccattttgcctcctttcacttctcacctttatcgacaacaccaacatggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgccgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctaggttacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaatgggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagtggcggtgctttcaaagatgtgatcacagagggtgatgggagacgttcatattgacttttagaaactacataactagctctaggcattgtattatctaaaataaacttctattaagccaaaagtgttctatctgtctagtttgccgttg

SEQ ID NO: 10 - DNA, 1962 bp, Capsicum Annum
  Capsicum Annum AHAS gene
atggcggctgcagcgccccctcctcctcctttcaccaaaaccctatctcattccccttcctcctccgccaaatcccccactcttctccctagatccaccttcccttttacccggccccttctccttatccaccgccgtcgtttcacggtcaacaatgtcatttccactaatcataacgtttccctttctcaaccacccgaaacatttattacccgttttgcccctgacgagccccgaaaaggctgcgacgttctcgtggaagcactcgaaagagaaggggttaccgacgtctttgcataccctggcggtgcttcagtggagattcatcaggcattgactcgttcgaatataatccggaatgtcctgccacgtcatgagcaaggtggcgtattcgctgccgagggttacgcgcgcgccaccgggttccctggtgtgtgcattgcgacatcagggccgggagctacgaatctcgttagcgggcttgcggatgcgttgttggatagtattccgatagtggctattacgggtcaggtgccgaggaggatgattggtacggatgcgtttcaggagactccgattgtcgaggtaactaggtctataacgaagcataattatcttgttatggatgtagaggatattcccagggttgttcgtgaggcatttttcctagcaaaatcggggaggcctggtccagttttgattgatgttcctaaggatattcagcagcaattggtgatacctaattgggatcagccaatgaggttgcctggttacatgtctaggttgcctaaattgcctaatgagatgcttttggaacaaattgttaggcttatttctgagtcaaagaagcctgttttgtatgtgggaggtgggtgttcgcagtcgagtgaggagttgagacgatttgtggagcttactggtattcccgttgcgactactttgatgggtcttggagcttttccaactggggatgacctttctcttcagatgttgggtatgcatggcactgtctatgctaattatgctgtcgatagtagtgatttgctgcttgcgtttggggtgaggtttgatgatagggttactggtaaattggaagcttttgctagccgtgcgaaaattgtccacattgacattgattcagctgagattggaaagaacaagcagcctcatgtttcaatttgtgcagatatcaagttggcattacagggtttgaattccatattggagggtaaagaagctaagctgaagaagttggacttttctgcttggaggcaggagttaaacgagcagaaagtgaagtacccattgaattataagacttttggtgatgccatccctccacaatatgctattcaggttctagatgagttaaccgacggaaatgccattgttagtactggtgtggggcaacaccagatgtgggctgcccagtactataagttcaaaaagccacgccaatggttgacatctggtggattaggagcaatgggatttggtttgcccgctgctataggtgcggctgttggaagacccggggagattgtggttgacattgatggtgatgggagttttatcatgaatgtgcaggagttagcaacaattaaggtggagaacctcccagttaagattatgttgctgaataatcaacacttgggaatggtggttcaatgggaggatcggttctacaaggctaacagagcacacacttacctgggtaatcctgcaaatgaggaagaaatctttcctaatatgttgaaatttgcagaggcttgtggcgtacctgctgcaagagtgacacacagggatgatgttagagctgccattcagaagatgttggacactcctggaccatacttgttggatgtgattgtaccgcatcaggagcacgttttacctatgattcccagtggtggtgctttcaaagatgtgattacwgagggtgatgggagatgttcccactga

SEQ ID NO: 11 - DNA, 4048 bp, artificial sequence
  description of artificial sequence synthetic DNA
gatattggagagtaaggaaggtaaactgaagttggatttttctgcttggaggcaggagttgatggagcagaaagtgaagcacccgttgaactttaaaacttttggtgatgctattcctccacaatatgctatccaggttctagatgagttaactaatgggaatgctattataagtactggtgtggggcaacaccagatgtgggctgctcaatactataagtacagaaagccacgccaatggttgacatctggtggattaggagcaatgggatttggtttacccgctgctattggtgcagctgtgggaagaccggatgaagttgtggttgacattgatggcgatggcagtttcatcatgaatgtgcaggagctggcaacaattaaggtggagaatctcccagttaagattatgttactgaataatcaacacttgggaatggtggttcaactcgaggatcggttctataaggctaacagagcacacacatacctggggaatccttctaatgaggcggaaatctttcctaatatgttgaaatttgcagaggcttgtggtgtacctgctgcaagggtgacacatagggatgatcttagagctgccattcagaagatgttagacactcctgggccatacttgttggatgtgattgtacctcatcaggaacatgttctacctatgattcccagtggcggagctttcaaagatgtgatcacagagggtgacgggagaatttcctattgagtttgagaagctgcagagctagttctaggcgtctaggccttgtattatctaaaataaacttctattaagccaaacatgttctgtctattagtttgttattagtttttgccgtggctttgctcatcgtcactgttgtactattaagtagttgatatttatgtttgttttgcatcatccccctttggttttgaatgtgaaggatttcagcaaagtttcatcctctatttgcaacaatctggagattaatttctaatggagtagtttagtgtaataaagactagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacaggatctatggctcctaagaagaagagaaaggttataacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagccgcggttcccgggagacctttagctagcttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaaattcgtaatcatggtcatagcatgcttggaacttcctctttaggttggatgtaatccctattagggctttctcttaattttattattgaattgttggcttttaatctgagcaagttgatttgcagctttctctcgagtcctaggagcaatacgttatctctgtctcctatttcctagtggataatcttatgatggaaatatgtggagttaggaaactgttgactgctaaatttctctttgtgaggcgtctgacaggtatgctttcaatctatagcagtttgatcagactttgtttacgtataacaatgttacgcaaacaaacacgtgctttttaaacagttataggtgcttagctaccgacaatacatcacatataacaggtacatgtatatctggcgttttgcttttaaatagtacatttcatttttgtattatgcactgaccagaccctgtttatggggtttgttgttgtgttattcactgaatctttaacattcaatcttcatgagaaactattctttacggcgtctaatgttctttctactaaacaaccaagtctttgtacctaacacacattgtaattgatcactagaaacttgtcaagttgctgatttagtaatctattttcttataatgaagatggaacttatcattcccaaaaatatatcctccttttgttttcaaggttacaaattctctagaaaatcatttcatgtggagtagctagtatctttaaacattaagtaattatctcctgagttctgcctgcctcttatatttctttggtgattcctctttttttaggggtgccgtgctaggggatattttttgtggagcaatccttttgcggaactacttatattcaatatattaagtattattggtttatttcttttaaaatccatatttgatttcacaaccataatcgggtaattcatgatacccatgaatatttctatcaaattcttaatgcttctatataagcacaattgtgattttactcgactttgagcatgtcttcaaagttgaaaatttaggtgtttcttgcatggtgttatagctgtcaaagtggtgttagggatgaaaagttttgcggatgagggagagctctgcatggcgtagaaggtcaccaaacatgtctcctctctctatttctactagcatcgcctagaagcctatcaatttgttgagaggacttatattaccgaggaagatacaaccgtttttaaagttaggaaaaaacattattcataagttatttactatggttctaggtgatcttggtccatcataatcaagttttcatcttcttaatttctctcatttttgctttggggtgtgtcttagttttcatcacaaagggaagaagatccattagagcatcacatgttctttgaacctaagacaagactctttatttaacccccgacacattatccttcaatgaagttttctcctagggagag

SEQ ID NO: 12 - DNA, 29 bp, artificial sequence
  description of artificial sequence synthetic oligonucleotide
atttccactatccaaaaagtttccgagac

SEQ ID NO: 13 - DNA, 29 bp, artificial sequence
  description of artificial sequence synthetic oligonucleotide
cgcgtattaaatgtataattgcgggactc